(19) United States
(12) Patent Application Publication
(10) Pub. No.: US 2025/0340954 A1
(43) Pub. Date: Nov. 6, 2025

(54) MODULATION OF ORAL MICROBIOTA IN PERIODONTAL DISEASE

(71) Applicant: MARS, INCORPORATED, Mclean, VA (US)

(72) Inventors: Lucy Jane HOLCOMBE, Melton Mowbray (GB); Corryn Victoria WALLIS, Melton Mowbray (GB)

(73) Assignee: MARS, INCORPORATED, Mclean, VA (US)

(21) Appl. No.: 18/854,204

(22) PCT Filed: Apr. 5, 2023
(86) PCT No.: PCT/US2023/017529

§ 371 Date: Oct. 4, 2024

Related U.S. Application Data
(60) Provisional application No. 63/327,585, filed on Apr. 5, 2022.

(51) Int. Cl. C12Q 1/689; A23K 50/42; C12Q 1/6806; C12Q 1/6869

(52) U.S. Cl. CPC C12Q 1/689; A23K 50/42; C12Q 1/6806; C12Q 1/6869

(57) ABSTRACT

Methods of modulating oral microbiota in companion animals with the administration of dry food forms are provided herein. The methods provide an avenue for identifying animals at risk of developing periodontal disease.

### CROSS-REFERENCE TO RELATED APPLICATIONS

This application is a U.S. National Stage patent application under 35 U.S.C. § 371 of International Patent Application No. PCT/US2023/017529, filed on Apr. 5, 2023, which claims priority to U.S. Provisional Patent Application Ser. No. 63/327,585, filed on Apr. 5, 2022, the contents of each of which are hereby incorporated by reference in their entireties, and to each of which priority is claimed.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Oct. 4, 2024, is entitled “069269.0611_ST26.xml”, and is 65,287 bytes in size.

### TECHNICAL FIELD

The presently disclosed subject matter relates to methods of modulating or managing the oral microbiota of animals, particularly of dogs, susceptible to or diagnosed with periodontal disease.

### BACKGROUND

Periodontal disease is one of the most common diagnoses in first opinion veterinary practice, with prevalence estimates based predominantly on visual assessment of dogs ranging from 7% to 20% (Lund et al., 1999; Robinson et al., 2016; O'Neill et al., 2014). These initial diagnoses underestimate the extent of the disease as more detailed assessments of the periodontium (gingiva, cementum, periodontal ligament and alveolar bone) under general anesthesia indicate that the prevalence within dogs is much higher (44% to 100%) (Butković et al., 2001; Harvey et al., 1994; Kyllar and Witter, 2005; Isogai et al., 1989; Kortegaard et al., 2008; Hoffman and Gaengler, 1996). The initial stage of periodontal disease is apparent as red and inflamed gingiva and, without effective treatment, this can progress to periodontitis.

Periodontitis is where the inflammation extends deeper into the periodontium causing irreversible damage. This can lead to periodontal abscesses, ulcers in the mucous membranes and is the principal cause of tooth loss in dogs (Harvey, 2005; Gorrel, 2013; Niemiec, 2008; Niemiec, 2012). Several studies also suggest there is an association between periodontitis and systemic disease (DeBowes et al., 1996; Pavlica et al., 2008; Glickman et al., 2009; Pereira Dos Santos et al., 2019).

Periodontal health can be maintained with an effective homecare regime combined with regular health checks, and veterinary treatment if required, by a veterinarian. A variety of homecare regimes have been shown to help prevent the build-up of plaque or bacterial communities on the tooth surface and these include tooth brushing, dental chews, dental diets and oral solutions or gels (Wallis and Holcombe, 2020). Some of these products work via mechanical abrasion resulting in the cleaning of the tooth and oral surfaces whereas others contain active ingredients.

Recently, it has been shown that feeding dental chews to dogs can shift the bacterial composition of dental plaque towards a profile associated with periodontal health (Oba et al., 2021; Ruparell et al., 2019). There is also some evidence indicating that dry diets help to prevent the build-up of plaque, bacterial communities, and calculus and reduce the levels of gingivitis compared to softer wet diets (Logan, 2006; Gawor et al., 2006). However, other studies have found no correlations between dietary consistency and the levels of plaque, bacterial communities, calculus or periodontal health status (Hoffman and Gaengler, 1996; Logan, 2006) (Harvey et al., 1996; Boyce and Logan, 1994).

Pet food products exist that offer some benefit to the oral health of animals; however, such foods generally work via mechanical action, i.e., food can affect changes in oral health by helping to keep an animal's teeth clean, through removal of plaque from the teeth by abrasion. Although such products can aid in the maintenance of the oral health of animals, there still exists a need to provide improved means for maintaining the oral health of animals and, in particular, for modulating the oral microbiota.

Accordingly, there exists a need for improved methods of modulating oral microbiota in animals, particularly to modulate, reduce or manage oral diseases or disorders (e.g., periodontal disease). The presently disclosed subject matter has advantageously identified that feeding a particular diet type to animals can have a significant effect on oral microbiota in order to promote a healthier microbiota and reduce the likelihood of the development of oral diseases or disorders (e.g., periodontal disease).

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages, and in accordance with the purpose of the disclosed subject matter, as embodied and broadly described, the disclosed subject matter includes in one aspect a method for modulating the oral microbiota in companion animals, wherein the method comprises feeding the companion animal a dry pet food.

In certain aspects, the present disclosure provides a method of modulating the oral microbiota in a companion animal, comprising feeding the animal a dry pet food. In certain embodiments, modulating the oral microbiota results in an increase in bacteria associated with good oral health. In certain embodiments, modulating the oral microbiota results in an increase in at least one bacterial taxa species selected from the group consisting of Capnocytophaga cynodegmi, Capnocytophaga canimorsus, Capnocytophaga sp. COT-295/FOT-311, Pasteurella dagmatis, Pasteurella stomatis, Pasteurella mulocida, Pasteurella sp. FOT-354, Bergeyella zoohelcum, Lachnospiraceae bacterium COT-106, SR1 bacterium COT-380, Lautropia sp. COT-175, Porphyromonas cangingivalis, Prevotella sp. COT-372, Brachymonas sp. COT-015, Stenotrophomonas sp. FOT-090, and a combination thereof.

In certain aspects, the present disclosure also provides a method of improving the oral health of an animal by modulating the animal's oral microbiota, comprising feeding the animal a dry pet food. In one aspect, the present disclosure provides a method of decreasing the formation of a disease-associated bacterial population on a biological surface of a companion animal in need thereof, comprising feeding the animal a dry pet food. In certain embodiments, the biological surface is an oral surface. In certain embodiments, the oral surface is a subgingival dental surface, a gingival margin dental surface, a supragingival dental surface from the cheek, or a surface of the tongue. In certain embodiments, the oral surface is a gingival margin dental surface, a subgingival dental surface, a supragingival dental surface, or a combination thereof. In certain embodiments, the oral surface is a gingival margin dental surface. In certain embodiments, the oral surface comprises an oral plaque.

In certain embodiments, the oral surface comprises at least one bacteria taxa order selected from the group consisting of Spirochaetales, Selenomonadales, Desulfovibrionales, Clostridiales, Fusobacteriales, Bacteroidales, Lactobacillies, Mycoplasmatales, Saccharimonadales, Unclassified WS6 (Dojkabacteria), and a combination thereof. In certain embodiments, the at least one bacteria taxa order is selected from the group consisting of Spirochaetales, Selenomonadales, Desulfovibrionales, Clostridiales, and a combination thereof.

In certain embodiments, the oral surface comprises at least one bacteria taxa family selected from the group consisting of Spirochaetaceae, Veillonellaceae, Desulfohalobiaceae, Peptostreptococcaceae, Mycoplasmataceae, Unclassified WS6 (Dojkabacteria), Streptococcaceae, Porphyromonadaceae, Lachnospiraceae, Saccharimonadaceae, and a combination thereof.

In certain embodiments, the oral surface comprises at least one bacteria taxa species selected from the group consisting of Treponema sp. COT-207, Treponema sp. COT-247, Schwartzia sp. FOT-014/COT-063, Treponema sp. COT-249, Unclassified Treponema, Treponema sp. COT-201, Treponema sp. FOT-142/COT-200, Desulfovibrionales bacterium COT-009, Treponema sp. COT-350, Peptostreptococcaceae bacterium COT-030/FOT-028, Peptostreptococcaceae sp. COT-033/FOT-053, and a combination thereof.

In certain aspects, the present disclosure further provides a method of identifying a companion animal at risk of periodontal disease, comprising a) obtaining an oral plaque sample from the animal; b) isolating a nucleic acid from the sample; c) analyzing the nucleic acid; and d) identifying at least one bacteria taxa present in the sample; wherein, if the at least one bacteria taxa is associated with disease, the animal is at risk of periodontal disease. In certain embodiments, the oral plaque sample comprises a subgingival dental plaque, a gingival margin dental plaque, a supragingival dental plaque, a bacterial sample from the cheek, a bacterial sample from the tongue, or a combination thereof. In certain embodiments, the oral plaque sample comprises a gingival margin dental plaque, a subgingival dental plaque, a supragingival dental plaque, or a combination thereof. In certain embodiments, the oral plaque sample is a gingival dental plaque sample. In certain embodiments, the nucleic acid comprises 16S rDNA.

In certain embodiments, the at least one bacteria taxa comprise a bacteria taxa order selected from the group consisting of Spirochaetales, Selenomonadales, Desulfovibrionales, Clostridiales, Fusobacteriales, Bacteroidales, Lactobacillies, Mycoplasmatales, Saccharimonadales, Unclassified WS6 (Dojkabacteria), and a combination thereof. In certain embodiments, the at least one bacteria taxa comprise a bacteria taxa family selected from the group consisting of Spirochaetaceae, Veillonellaceae, Desulfohalobiaceae, Peptostreptococcaceae, and a combination thereof. In certain embodiments, the at least one bacteria taxa comprise a bacteria species selected from the group consisting of Treponema sp. COT-207, Treponema sp. COT-247, Schwartzia sp. FOT-014/COT-063, Treponema sp. COT-249, Unclassified Treponema, Treponema sp. COT-201, Treponema sp. FOT-142/COT-200, Desulfovibrionales bacterium COT-009, Treponema sp. COT-350, Peptostreptococcaceae bacterium COT-030/FOT-028, Peptostreptococcaceae sp. COT-033/FOT-053, and a combination thereof.

In certain embodiments, the methods further comprise feeding the animal a dry pet food.

In certain embodiments, the companion animal is a dog. In certain embodiments, the dog is a toy breed, a small breed, a medium breed, a large breed, or a giant breed. In certain embodiments, the dog is selected from the group consisting of Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy, and Yorkshire Terrier. In certain embodiments, the dog is a Yorkshire Terrier. In certain embodiments, analyzing the DNA comprises sequencing the nucleic acid.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the kits and methods of the disclosed subject matter. Together with the description, the drawings serve to explain the principles of the disclosed subject matter.

### BRIEF DESCRIPTION OF THE DRAWINGS

The present disclosure will now be described in detail, by way of example only, with reference to the drawings, in which:

FIG. 1A and FIG. 1B provide a non-metric multidimensional scaling with dimensions labelled by sample type (FIG. 1A); and diet type (FIG. 1B).

FIG. 2 provides a stacked bar chart showing the phylum level composition (%) of gingival margin plaque and subgingival plaque when dogs are fed wet commercial diets (wet), dry commercial diets (dry) and mixture of the two (mixed).

FIG. 3 provides boxplots of the scores for factor 3 for gingival margin and subgingival plaque for each diet group: commercial dry, commercial wet, mixture commercial wet and dry. Individual dots represent outliers.

FIG. 4 provides boxplots of the scores for factor 7 for gingival margin and subgingival plaque for each diet type: commercial dry, commercial wet, mixture commercial wet and dry. Individual dots represent outliers.

FIG. 5 provides the Shannon diversity of gingival margin and subgingival plaque samples when dogs were fed a dry commercial diet, wet commercial diet or a mixture of the two. Single dot indicates average Shannon Diversity, and bars represent 95% confidence intervals.

FIG. 6 provides a summary of 26 species that were present at a relative abundance >1.0%, together accounting for 58.2% of total sequence reads. The Canine Oral Taxon numbers (COT) and Feline Oral Taxon numbers (FOT) are provided for the species listed if available.

FIGS. 7A-7F provide a summary of OTUs of factor 3 associated with changes in microbiota composition between wet and dry commercial diets in GM plaque.

FIG. 8 provides a summary of mean gingivitis scores and proportion of periodontitis teeth across the different diet groups.

FIG. 9 provides a summary of 16s rDNA sequences of OTUs most influential in factor 3. SEQ ID Nos: 1-38 are provided from top to bottom, respectively.

### DETAILED DESCRIPTION

Reference will now be made in detail to the various exemplary embodiments of the disclosed subject matter, exemplary embodiments of which are illustrated in the accompanying drawings. The presently disclosed subject matter relates to edible products and related methods of modulating an oral microbiota of animals. The presently disclosed subject matter is particularly suited for modulating the microbiota of a companion animal, e.g., a domestic dog.

### A. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the methods and compositions of the present disclosure and how to make and use them.

Throughout the description and claims of this specification, the words “comprise” and “contain” and variations of the words, for example “comprising” and “comprises,” mean “including but not limited to,” and do not exclude other components, integers or steps. Moreover, the singular encompasses the plural unless the context otherwise requires. In particular, where the indefinite article is used, the specification is to be understood as contemplating plurality as well as singularity, unless the context requires otherwise.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

The term “animal” as used in accordance with the present disclosure refers to a wide variety of animals, such as quadrupeds, primates, and other mammals. For example, the term “animal” can refer to domestic animals including, but not limited to, dogs, cats, horses, cows, ferrets, rabbits, pigs, rats, mice, gerbils, hamsters, goats, and the like. The term “animal” can also refer to wild animals including, but not limited to, bison, elk, deer, venison, duck, fowl, fish, and the like. In certain embodiments, the animal is a companion animal. In certain instances, the animal is a domestic dog or cat.

The terms “animal feed,” “animal feed compositions,” “pet food,” “pet food article,” “pet food product”, “edible product” or “pet food composition” are used interchangeably herein and refer to a composition intended for ingestion by an animal or pet. Any composition intended for ingestion by an animal or pet is suitable for use with the present disclosure. Such compositions can include kibble or dry foods, moist or wet foods, semi-moist foods, frozen or freeze-dried foods, raw foods, or combinations thereof. Compositions of the present disclosure can be used, for example, as a main meal, a meal supplement, a treat, or a combination thereof. The compositions can be nutritionally balanced. In alternate embodiments, the compositions are not nutritionally balanced. For example, and not by way of limitation, pet foods can include, without limitation, nutritionally balanced compositions suitable for daily feed, such as kibbles, as well as supplements and/or treats, which can be nutritionally balanced. In an alternative embodiment, the supplement and/or treats are not nutritionally balanced. In certain aspects, the supplement can include a topper.

The terms “chew” or “oral chew” refers an edible product that in some instances, in one aspect, can be differentiated from food by virtue of its nutritional content. Specifically, a conventional dog “food” is nutritionally complete and provides the full range of the dog's daily nutrition requirements. It is also intended to be the major source of the dog's calorific intake, A chew need not provide such nutrition or calorific content. A chew can further be distinguished from a “food” with regard to its size. The largest pieces in a food product are generally smaller than the size of a chew. A chew can be further distinguished with regard to the time taken to consume a piece of chew compared to a piece of food. Normally the consumption time for a piece of chew is much longer than a piece of food. A piece of food can generally be consumed in less than 10 seconds by an average sized dog, whereas a chew would take at least 20 seconds for an average-sized dog to consume. For example, and not by way of limitation, the edible chew product can be moulded, aerated, or extruded.

The phrase “expected microbiota” can refer to the actual microbiota found before the administration of the edible product. In one embodiment, the expected microbiota can refer to the actual microbiota found before the administration of the edible product and before any method has been used to clean the animal's mouth such as a scale and polish. Alternatively, it can refer to the predicted microbiota, based on microbiota found in other animals of the same or similar species or breeds.

The terms “nutritionally balanced” or “nutritionally complete” in reference to a composition means that the composition, such as pet food, has known required nutrients to sustain life in proper amounts and proportion based on recommendations of recognized authorities, including governmental agencies, such as, but not limited to, United States Food and Drug Administration's Center for Veterinarian Medicine, the American Feed Control Officials Incorporated, National Research Council (NRC), and The European Pet Food Industry (FEDIAF) guidelines (e.g., www.fediaf.org/images/FEDIAF_Nutritional_Guidelines_2019_Update_030519.pdf), in the field of pet nutrition, except for the additional need for water.

As used herein, “kibble,” “dry kibble,” “dry food,” “dry composition,” or “dry pet food” refers to a pet food product with a moisture level less than or equal to 15%, by weight of the food product. A kibble can be nutritionally balanced and complete. In general, such dry food can even contain much less than about 15% of moisture content, relative to the total weight of the composition, such as from about 1 to about 15% of moisture content. The term “semi-moist,” as used herein, refers to a food product with a moisture level between 15% and 50%, by weight of the food product. The term “wet,” as used herein, refers to a food product having a moisture content equal to or greater than 50%, by weight of the food. Kibbles can range in texture from hard to soft. Kibbles can range in internal structure from expanded to dense. Kibbles can be formed by an extrusion process. For instance, a kibble can be formed from a core and a coating to form a kibble that is coated, also called a coated kibble. It should be understood that when the term “kibble” is used, it can refer to an uncoated kibble or a coated kibble. The moisture content of a dry food composition of the disclosure can be measured according to any methods known in the art. Although this definition is not limited to one specific form of presentation, a dry food or dry composition is generally presented in the form of (biscuit-like) kibbles, and/or dry core components. For instance, a dry food can be manufactured by mixing together ingredients and kneading in order to make consistent dough that can be cooked. The process of creating a dry pet food is usually done by baking and/or extruding. The dough is typically fed into a machine called an expander and/or extruder, which uses pressurized steam or hot water to cook the ingredients. While inside the extruder, the dough is under extreme pressure and high temperatures. The dough is then pushed through a die (specifically sized and shaped hole) and then cut off using a knife. The puffed dough pieces are made into a dry product, such as a kibble, by passing it through a dryer so that moisture is dropped down to a defined target ensuring stability of the food until consumption. The product/kibble can then be sprayed with fats, oils, minerals, vitamins, natural extracts cocktail, flavors and optionally sealed into packages.

As used herein, the term “extrude” means an animal feed that has been processed by, such as by being sent through, an extruder. In one embodiment of extrusion, kibbles are formed by an extrusion processes wherein raw materials, including starch, can be extruded under heat and pressure to form the pelletized kibble form, which can be a core. Any type of extruder can be used, non-limiting examples of which include single screw extruders and twin-screw extruders.

As used herein, the term “aerated” refers to the incorporation of a gas into a food material. For purposes herein, the gas is not particularly limited, and can be, for example, air, nitrogen, carbon dioxide, and gas combinations thereof. Aerated means an expanded kibble with numerous air bubbles internally, and the internal “aeration” (the texture itself) can be measured by porosity.

As used herein, the term “oral disease or disorder,” refers to a disease or disorder that occurs in an oral cavity of a subject (e.g., an animal) and that is caused by or is associated with one or more bacteria. For example, the disease or disorder can affect the teeth or the gums of the subject. Exemplary oral diseases or disorders of the present disclosure include, but are not limited to, periodontal disease, caries, gingival stomatitis, odontoclastic resorptive lesions, and oral malodor.

The term “oral microbiota” refers to the microorganisms found in the oral cavity. In particular, it can refer to the bacteria, archaea, fungi, bacteriophage, and protozoa found in the oral cavity, and more specifically to bacterial composition or bacterial communities of dental plaque or oral plaques. It can refer to plaque above (supragingival) and/or below the gum line (subgingival), and/or where the gum joins the tooth (gingival margin) plaque, or plaques present in the mouth such as on the tongue or cheek, or bacteria in the saliva.

As used herein, the term “periodontal disease,” also known as gum disease, refers to an inflammation or infection that affect the tissues surrounding the teeth. Periodontal disease can range in severity, e.g., from gingivitis (e.g., dental plaque-induced gingivitis) to periodontitis (e.g., clinical attachment and alveolar bone loss).

As used herein, the term “plaque” refers to an extracellular matrix comprising one or more microorganisms such as, but not limited to, bacteria, fungi, algae, archaea, bacteriophage, and protozoa, which is attached to a surface. As used herein, the term plaque includes bacterial communities. For example, but not by way of limitation, such surfaces can include tooth, mucosal, apatitic, bone and abiotic (e.g., implant, dentures, etc.) surfaces. Plaques can form on living or non-living surfaces and can exist in natural and industrial settings. In certain embodiments, plaques are present within the oral cavity. For example, but without any limitation, plaques can be present on the surface of teeth, on the surface of mucosal/soft-tissues such as gingivae/periodontium and inside a tooth canal (e.g., endodontic canal). In one embodiment, the term plaque refers to bacteria.

As used herein, the term “taxa” refers to taxonomical groups, for example, kingdom, phylum, class, order, family, genus, and species. As used herein, the term “abundance” can refer to an absolute amount of given bacterial taxa present within a sample. For example, but without any limitation, an abundance can refer to the count of bacterial sequences of bacterial taxa after appropriate amplification of 16S rDNA. In another example, without any limitation, an abundance can refer to the count of bacterial sequences of bacterial taxa after appropriate amplification of a gene such as rpoB, tuf, gyrA or gyrB, sodA, heat shock proteins, ITS1, ITS2, and/or 28s rDNA. In certain non-limiting embodiments, the abundance of a given bacteria taxa can be determined by molecular methods (e.g., 454 pyrosequencing, polymerase chain reaction (PCR), quantitative PCR (qPCR), 16S rDNA amplicon sequencing, shotgun sequencing, metagenome sequencing, Illumina sequencing, PacBio sequencing, or nanopore sequencing).

As used herein, the term “relative abundance” can refer to a percentage composition of bacteria of particular bacterial taxa (e.g., species) relative to the total number of bacteria in the sample. In certain embodiments, the relative abundance can be calculated by determining the number of sequences of given bacterial taxa divided by the total number of all bacterial sequences which is then multiplied by 100. For example, but without any limitation, the relative abundance can refer to the amounts and relative amounts of nucleic acid present in a sample after appropriate amplification of 16S rDNA.

As used herein, the term “modulating” refers to changing or regulating the presence or prevalence of particular microorganisms in the oral microbiota. In certain embodiments, the oral microbiota can be modulated by decreasing the number of bacteria taxa species associated with periodontal disease. In certain embodiments, the oral microbiota can be modulated by increasing the number of bacteria taxa species associated with good oral health. As used herein, the term modulating also includes regulating or maintaining presence or prevalence of particular microorganisms in the oral microbiota. In certain embodiments, the phrase “modulating the oral microbiota” refers to causing the oral microbiota population to change (i.e., increasing or decreasing), compared to the oral microbiota that would be expected to be found if the animal had not been fed the edible product of the present disclosure. Alternatively, “modulating the oral microbiota” refers to regulating or maintaining the oral microbiota population. Modulation of the oral microbiota can comprise promoting health-associated oral cavity flora.

As used herein, and as is well-understood in the art, “treatment” refers to an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment. The term “preventing,” as used herein, means partially or completing treating before the disorder or condition occurs.

As used herein, the term “weight percent” is meant to refer to the quantity by weight of a constituent or component, for example, in the pet food composition as a percentage of the overall weight of the pet food composition. The terms “weight percent,” “wt-%,” “wt. %”, and “wt %” are used interchangeably.

Preferred features of each aspect of the presently disclosed subject matter can be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, can be taken independently or in any combination. That is, all embodiments and/or features of any embodiment can be combined in any way and/or combination, unless such features are incompatible.

### B. Bacteria in the Oral Microbiota/Microbiome

In certain embodiments, the bacteria taxa associated with periodontal disease include a bacterium from the Spirochaetes phylum. In certain embodiments, the bacteria taxa associated with periodontal disease include a member of the genus Treponema. In certain embodiments, the bacteria taxa associated with periodontal disease include Treponema sp. COT-207 (OTU #8534), Treponema sp. COT-247 (OTU #11699), Treponema sp. COT-249 (OTU #5093), Treponema sp. COT-201 (OTU #10686), and/or Treponema sp. COT-350 (OTU #4245.

In certain embodiments, the bacteria taxa associated with periodontal disease include a bacterium from the Firmicutes phylum. In certain embodiments, the bacteria taxa associated with periodontal disease include Schwartzia sp. FOT-014/COT-063 (OTU #10845), Selenomonas sp. COT-167 (OTU #9061), Mycoplasma equirhinis, Unclassified WS6 (Dojkabacteria), Streptococcus fryil Streptococcus sp. COT-297, Porphyromonas sp. COT-181, Treponema sp. COT-359/FOT-207, Treponema sp. COT-397, Lachnospiraceae bacterium FOT-021, Candidatus saccharimonas, Peptostreptococcaceae bacterium COT-030/FOT-028 (OTU #8371), and/or Peptostreptococcaceae sp. COT-033/FOT-053 (OUT #6529).

In certain embodiments, the bacteria taxa associated with periodontal disease include a bacterium from the Proteobacteria phylum. In certain embodiments, the bacteria taxa associated with periodontal disease include Desulfovibrionales bacterium COT-009 (OTU #7737).

Bacterial community profiles within an oral microbiome of an animal can vary depending on the source of a sample taken from the animal. For example, three discrete oral niches can include soft tissue surfaces, such as the lip, cheek, and tongue; hard tissue surfaces, such as the teeth; and saliva. In some embodiments, the oral niche is from a hard tissue surface, such as one or more teeth. In some embodiments, the oral niche includes the gingival margin or supragingival surface.

In certain embodiments, the oral microbiota can be modulated by increasing or decreasing the presence or prevalence of particular microorganisms, particularly bacteria taxa or groups of bacteria taxa. In certain embodiments, the oral microbiota can be modulated by increasing the presence of particular microorganisms while decreasing the presence of certain other microorganisms, particularly bacteria taxa or groups of bacteria taxa. For example, and not by way of limitation, bacteria taxa can include Actinobacteria, Epsilonbacteraeota, Synergistetes, SR1, Chlorobi, Tenericutes, Firmicutes, Bacteroidetes, Proteobacteria, Fusobacteria, Patescibacteria, Spirochaetota/Spirochaetes, or combinations thereof.

In certain embodiments, the oral microbiota can be modulated by increasing the presence or prevalence of bacteria taxa selected from the group consisting of Capnocytophaga cynodegmi, Capnocytophaga canimorsus, Capnocytophaga sp. COT-295/FOT-311, Pasteurella dagmatis, Pasteurella stomatis, Pasteurella mulocida, Pasteurella sp. FOT-354, Bergeyella zoohelcum, Lachnospiraceae bacterium COT-106, SR1 bacterium COT-380, Lautropia sp. COT-175, Porphyromonas cangingivalis, Prevotella sp. COT-372, Brachymonas sp. COT-015, Stenotrophomonas sp. FOT-090, Moraxella sp. FOT-350, Actinomyces canis, Fusobacterium sp. FOT-120, Unclassified Neisseria, Frederiksenia canicola, Alloprevotella sp. FOT-167, and a combination thereof.

In certain embodiments, the oral microbiota can be modulated by decreasing the presence or prevalence of bacteria taxa selected from the group consisting of Treponema sp. COT-207, Treponema sp. COT-247, Schwartzia sp. FOT-014/COT-063, Selenomonas sp. COT-167, Treponema sp. COT-249, Unclassified Treponema, Treponema sp. COT-201, Treponema sp. FOT-142/COT-200, Desulfovibrionales bacterium COT-009, Treponema sp. COT-350, Peptostreptococcaceae bacterium COT-030/FOT-028, Peptostreptococcaceae sp. COT-033/FOT-053, Mycoplasma equirhinis, Unclassified WS6 (Dojkabacteria), Streptococcus fryil Streptococcus sp. COT-297, Porphyromonas sp. COT-181, Treponema sp. COT-359/FOT-207, Treponema sp. COT-397, Lachnospiraceae bacterium FOT-021, Candidatus saccharimonas, and a combination thereof.

The oral microbiota can be modulated by increasing the number of bacteria taxa species associated with good oral health, particularly species strongly associated with good oral health in a companion animal to which the edible product or the pet food (e.g., dry pet food) is administered, compared to the expected microbiota in that companion animal, if the edible product or the pet food (e.g., dry pet food) was not administered. In certain embodiments, the oral microbiota can also be modulated by increasing the prevalence, i.e., the number present, of one or more bacteria taxa associated with good oral health, particularly one or more bacteria taxa strongly associated with good oral health. In certain embodiments, the oral microbiota can also be modulated by increasing the ratio of bacteria taxa associated with good oral health to bacteria or bacterial species associated with poor oral health or disease in the oral microbiota.

In certain embodiments, the bacteria taxa associated with good oral health include a bacterium from the Bacteroidetes phylum. In certain embodiments, the bacteria taxa associated with good oral health include a bacterium from the Firmicutes phylum. In certain embodiments, the bacteria taxa associated with good oral health include a bacterium from the Patescibacteria phylum. In certain embodiments, the bacteria taxa associated with good oral health include a bacterium from the Proteobacteria phylum. In certain embodiments, the bacteria taxa associated with periodontal disease include a species selected from the group consisting of Capnocytophaga cynodegmi, Capnocytophaga canimorsus, Capnocytophaga sp. COT-295/FOT-311, Pasteurella dagmatis, Pasteurella stomatis, Pasteurella mulocida, and Pasteurella sp. FOT-354, Bergeyella zoohelcum, Lachnospiraceae bacterium COT-106, SR1 bacterium COT-380, Lautropia sp. COT-175, Porphyromonas cangingivalis, Prevotella sp. COT-372, Brachymonas sp. COT-015, and Stenotrophomonas sp. FOT-090.

In certain aspects, the oral microbiota can be modulated by decreasing the number of bacteria taxa associated with poor oral health or with disease, particularly species strongly associated with poor oral health or disease in an animal to which the edible product or the pet food (e.g., dry pet food) is administered, compared to the expected microbiota in that animal, if the edible product or the pet food (e.g., dry pet food) was not administered. In certain embodiments, the oral microbiota can also be modulated by decreasing the prevalence or relative proportion, i.e., the number present, of one or more bacteria taxa associated with poor oral health or disease, particularly one or more bacteria taxa strongly associated with poor oral health or disease.

Non-limiting examples of bacteria taxa associated or strongly associated with poor oral health or disease include Treponema sp. COT-207, Treponema sp. COT-247, Schwartzia sp. FOT-014/COT-063, Selenomonas sp. COT-167, Mycoplasma equirhinis, Unclassified WS6 (Dojkabacteria), Streptococcus fryil Streptococcus sp. COT-297, Porphyromonas sp. COT-181, Treponema sp. COT-359/FOT-207, Treponema sp. COT-397, Lachnospiraceae bacterium FOT-021, Candidatus saccharimonas, Treponema sp. COT-249, Unclassified Treponema, Treponema sp. COT-201, Treponema sp. FOT-142/COT-200, Desulfovibrionales bacterium COT-009, Treponema sp. COT-350, Peptostreptococcaceae bacterium COT-030/FOT-028, and Peptostreptococcaceae sp. COT-033/FOT-053.

### C. Edible Pet Foods Products

Modulating the oral microbiota can result in improved oral health, for example by lowering the likelihood of the animal developing periodontal disease. The present disclosure provides different methods of modulating the oral microbiota of animals with the administration of edible pet food products in different forms. In particular embodiments, the oral microbiota of animals is modulated by administering specific food diet forms to the animal. In particular embodiments, the diet form comprises a dry food diet, a wet food diet, or a combination of a dry food and wet food diet. In certain embodiments, the diet form is a dry food diet. In certain embodiments, the diet form is a wet food diet. In certain embodiments, the diet form is a combination of a dry food and wet food diet. Any of the diet forms can be commercially available pet food products.

In the presently disclosed subject matter, the pet food product can be administered alone to modulate oral microbiota over time. Alternatively, the pet food product can be used to supplement existing oral health and hygiene practices. Such practices include, but are not limited to, tooth brushing, dental chews, and oral solutions or gels.

The food product can be a complete animal feed, i.e., a feed that provides all the usual dietary requirements. Alternatively, the edible product can be a supplement to the animal's usual diet. In certain embodiments, the edible product can be nutritionally complete.

Any food product intended for ingestion by an animal or pet is suitable for use with the present disclosure. Such compositions can include kibble or dry foods, moist or wet foods, semi-moist foods, frozen or freeze-dried foods, raw foods, or combinations thereof. Food products of the present disclosure can be used, for example, as a main meal, a meal supplement, a treat, or a combination thereof. The food product can be nutritionally balanced. In alternate embodiments, the food products are not nutritionally balanced. For example, and not by way of limitation, the food products can include, without limitation, nutritionally balanced compositions suitable for daily feed, such as kibbles, as well as supplements and/or treats, which can be nutritionally balanced. In an alternative embodiment, the supplement and/or treats are not nutritionally balanced.

### D. Dog Breeds

The present disclosure relates to, inter alia, edible products and related methods for modulating the oral microbiota of an animal. The one or more bacteria modulated can be associated with an oral disease or disorder, e.g., periodontal disease, or good oral health. Size category of a dog can provide a general indication of its susceptibility to periodontal disease, with dogs in smaller size categories being generally more susceptible to periodontal disease than others. As used herein, the expression “size category” refers to the definition of the dog in terms of the average weight of the particular dog breed. Dogs of the same breed have relatively uniform physical characteristics, such as size and behavior.

The dog can be any breed of dog, including extra-small/toy, small, medium, large and extra-large/giant breeds. Medium-sized dogs can be further categorized into medium-small and medium-large sizes. Dog size categorizations by weight are known in the art, including by way of example, in Salt et al. (2017). Non-limiting examples of toy breeds include Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy, and Yorkshire Terrier. Examples of small breeds include, but are not limited to, French Bulldog, Beagle, Dachshund, Pembroke Welsh Corgi, Miniature Schnauzer, Cavalier King Charles Spaniel, Shih Tzu, and Boston Terrier. Examples of medium dog breeds include, but are not limited to, Bulldog, Cocker Spaniel, Shetland Sheepdog, Border Collie, Basset Hound, Siberian Husky, and Dalmatian. Examples of large breed dogs include, but are not limited to, Great Dane, Neapolitan mastiff, Scottish Deerhound, Dogue de Bordeaux, Newfoundland, English mastiff, Saint Bernard, Leonberger, and Irish Wolfhound. Cross-breeds can generally be categorized as toy, small, medium, and large dogs depending on their body weight. In certain embodiments, the dog is a toy breed. In certain embodiments, the dog is a medium, large or giant breed. Categories are defined as follows in Table 1.

TABLE 1

| Size Category name | Weight range of dog breed | |
|---|---|---|
| Toy/Extra small | Up to 6.5 | Kg |
| Small | 6.5-9 | Kg |
| Medium-small | 9-15 | Kg |
| Medium-large | 15-30 | Kg |
| Large | 30-40 | Kg |
| Giant | Over 40 | Kg |

The Fédération Cynologique Internationale currently recognizes 346 pure dog breeds. In certain embodiments, the breed of a dog can be identified, for example, either by observing its physical traits or by genetic analysis. A pedigree dog is the offspring of two dogs of the same breed, which is eligible for registration with a recognized club or society that maintain a register for dogs of that description. There are a number of pedigree dog registration schemes, of which the Kennel Club is the most well-known.

### E. Administration of Food Products

The methods of the disclosed subject matter can include administering or feeding an edible pet food product to an animal to modulate the oral microbiota of the animal. The methods of the disclosed subject matter are particularly well suited for use with companion animals, such as dogs, cats, and other domesticated animals.

In certain embodiments, the pet food can be administered to an animal with a clean mouth, e.g., after it has received a scale and polish. Without being bound by the theory, the edible product is thought to modulate, reduce or manage the formation of mature plaque due to its abrasive properties.

In certain embodiments, the pet food can be administered daily or more than once daily to the animal. In certain embodiments, the pet food can be administered to the animal daily, twice weekly, weekly or fortnightly. In certain embodiments, the pet food can be administrated to the animal at least 5, 10, 15, 20, 25, 30, 45, 60, 90 or 120 times. In certain embodiments, a supplement (e.g., a topper) can be administered to the animal food. In particular embodiments, the pet food can be administered to the animal at least 5 times. In certain aspects, the pet food can be administered to an animal with a clean mouth, e.g., after it has received a scale and polish or that has had its teeth cleaned, or generally having healthy gingiva. Without being bound by the theory, the edible product is thought to modulate, reduce or manage the formation of mature plaque due to its abrasive properties.

In certain aspects, the methods described herein can be carried out in conjunction with additional methodologies for improving oral health, including for example, cleaning the animal's teeth or mouth through other means such as brushing or rinsing.

In alternative embodiments, where the pet food product includes a wet pet food, the microbiome can be monitored to check for early signs of periodontal disease. In such instances, adjustments to the pet food form to include additional dry pet food to aid in the modulation of the bacterial composition towards bacteria associated with good oral health.

The present disclosure provides methods for managing formation of a plaque on a biological surface of a companion animal. In certain embodiments, the method comprises feeding the animal a dry pet food. In certain embodiments, the biological surface is an oral surface. Non-limiting example of oral surface include subgingival dental surface, gingival margin dental surface, supragingival dental surface, the cheek, or surface of the tongue. In certain embodiments, the oral surface is a gingival dental surface, a subgingival surface, or a supragingival dental surface. In certain embodiments, the oral surface is a gingival dental surface. In certain embodiments, the oral surface is a gingival margin dental surface. In certain embodiments, the oral surface comprises an oral plaque. In certain embodiments, the oral plaque on the surface comprises any of the bacteria taxa disclosed herein. For example, without any limitation, the oral surface plaque can include any of bacteria taxa species selected from the group consisting of Treponema sp. COT-207, Treponema sp. COT-247, Schwartzia sp. FOT-014/COT-063, Selenomonas sp. COT-167, Mycoplasma equirhinis, Unclassified WS6 (Dojkabacteria), Streptococcus fryil Streptococcus sp. COT-297, Porphyromonas sp. COT-181, Treponema sp. COT-359/FOT-207, Treponema sp. COT-397, Lachnospiraceae bacterium FOT-021, Candidatus saccharimonas, Moraxella sp. FOT-350, Actinomyces canis, Fusobacterium sp. FOT-120, Unclassified Neisseria, Frederiksenia canicola, Alloprevotella sp. FOT-167, Treponema sp. COT-249, Unclassified Treponema, Treponema sp. COT-201, Treponema sp. FOT-142/COT-200, Desulfovibrionales bacterium COT-009, Treponema sp. COT-350, Peptostreptococcaceae bacterium COT-030/FOT-028, Peptostreptococcaceae sp. COT-033/FOT-053, Capnocytophaga cynodegmi, Capnocytophaga canimorsus, Capnocytophaga sp. COT-295/FOT-311, Pasteurella dagmatis, Pasteurella stomatis, Pasteurella mulocida, Pasteurella sp. FOT-354, Bergeyella zoohelcum, Lachnospiraceae bacterium COT-106, SR1 bacterium COT-380, Lautropia sp. COT-175, Porphyromonas cangingivalis, Prevotella sp. COT-372, Brachymonas sp. COT-015, Stenotrophomonas sp. FOT-090, and a combination thereof.

The present disclosure provides kits that are useful in the modulation of the oral microbiota of an animal, e.g., to improve oral health of the animal. The kits can be used to administer and track administration of diets. In certain embodiments, the kits can be used to collect a sample in which one or more bacteria associated with an oral disease or disorder (e.g., periodontal disease) or good oral health are detected. A kit for modulating the oral microbiota of an animal can generally include, amongst other things, sample collection devices for collection of samples, one or more food compositions such as diets or supplements for administering to the animal, and instructions with respect to appropriate diet regimens for the animal.

### F. Methods of Detection and Quantification of Bacteria Taxa

The present disclosure includes methods for identifying a companion animal at risk of periodontal disease. In certain embodiments, the method comprises obtaining an oral plaque sample from the animal. In certain embodiments, the sample is obtained from the oral cavity. In certain embodiments, the sample from the oral cavity of the animal comprises oral plaque (e.g., subgingival dental plaque, gingival margin dental plaque, supragingival dental plaque or microorganisms from the cheek and/or tongue). In certain embodiments, the sample can comprise gingival margin dental plaque, subgingival dental plaque, and/or supragingival dental plaque. In certain embodiments, the sample can be fresh, frozen, or stabilized by other means such as the addition to preservation buffers or by dehydration using techniques such as freeze-drying.

After collecting the sample, the sample can be processed to extract nucleic acids (e.g., DNA and/or RNA). Non-limiting examples of techniques for isolating nucleic acids include the Qiagen DNeasy kit™, Qiagen QIAamp Cador Pathogen Mini kit™, the Nucleospin 96 Tissue kit (Macherey-Nagel), Qiagen DNeasy PowerSoil Pro, QIAzol Lysis Reagent, Qiagen RNeasy kit, Qiagen TurboCapture mRNA kit, and the Epicentre Masterpure Gram Positive DNA Purification Kit as well as Isopropanol DNA Extraction.

In certain embodiments, the methods disclosed herein comprise detection and quantification of bacteria, fungi, protozoa, and archaea. In certain embodiments, the detection and quantification of bacteria taxa include isolating DNA from the sample and sequencing the DNA. In certain embodiments, the detection and quantification of bacteria taxa include isolating DNA from the sample and quantifying the DNA (e.g., quantitative PCR).

Any suitable technique for detecting and quantifying bacterial taxa can be employed. Examples of techniques for detecting and quantifying bacterial taxa include, but are not limited to, 454 pyrosequencing, polymerase chain reaction (PCR), quantitative PCR (qPCR), 16S rDNA amplicon sequencing, shotgun sequencing, metagenome sequencing, Illumina sequencing, PacBio sequencing, and nanopore sequencing. In certain non-limiting embodiments, the bacterial taxa (e.g., species) can be determined by qPCR amplification and sequencing of the 16S rDNA. Other examples include shotgun sequencing to determine characteristic non-16SrDNA gene sequences or other metabolites and biomarkers for identification of the taxa.

In certain embodiments, the bacterial taxa can be determined by sequencing one or more of the variable regions of the 16S rDNA (e.g., V1-V3, V3-4, or V4). Non-limiting examples of sequencing methods including 454-pyrosequencing or Illumina sequencing. For example, but without any limitation, this process can include using universal bacterial primers 319F and 806R. In certain embodiments, these primers can be used for both amplification and sequencing.

The bacterial taxa can also be detected by other techniques such as RNA sequencing, protein sequence homology, or other biological markers indicative of the bacterial taxa. The sequencing data can then be used to determine the abundance or relative abundance of bacterial taxa in the sample. For example, the sequences can be clustered at 98%, 99%, or 100% identity and abundant taxa (e.g., those representing more than 0.001%, 0.005%, or 0.01% of the total sequences) can then be assessed for their relative proportions. Examples of techniques include, but are not limited to, logistic regression, partial least squares discriminate analysis (PLSDA), random forest analysis, and other univariate and multivariate methods.

In certain embodiments, the methods disclosed herein comprise identifying at least one bacteria taxa present in the sample. In certain embodiments, identifying at least one bacteria taxa present in the sample comprise annotating observed taxonomic units. For example, but without any limitation, annotation can be done using the basic local alignment search tool (BLAST). Depending on how precisely the alignment matches the top hit, an OTU can be allocated a suitable taxonomic assignment. As an example, if the alignment matches the top hit (e.g., top BLAST hit) with ≥98% sequence identity and ≥98% sequence coverage then a species level is assigned. If these criteria are not met, the next appropriate level of taxonomic assignment is allocated, for example, ≥94% genus, ≥92% family, ≥90% order, ≥85% class, or ≥80% phyla. This means that the various OTUs in a sample can be assigned to a species, a genus, a family, an order, a class, or a phyla. These are collectively referred to herein as “bacterial taxa”.

### EXAMPLES

For purpose of understanding and not limitation, the presently disclosed subject matter will be better understood by reference to the following Example, which is provided as exemplary of the disclosed subject matter, and not by way of limitation.

### Example 1: Assessment of Diet Type on Bacterial Composition of Canine Dental Plaque

Periodontal disease is common in dogs and is initiated by the build-up of plaque on the tooth surface. Management of plaque formation through an effective homecare regimen, such as tooth brushing or the provision of dental chews, is an effective means of maintaining healthy gingiva. The present Example assessed the impact of dry diets versus wet diets in the modulation or management of build-up of plaque. The primary objective of this study was to determine whether diet format influences the microbial composition of subgingival (SG) and gingival margin (GM) plaque.

### Methods

### Study Cohort

The 28 Yorkshire terriers (7 litters) investigated in this study were a subset of a larger study to determine the incidence of gingivitis and periodontitis in Yorkshire terriers (Wallis et al., 2019). The dogs were housed at the Waltham Petcare Science Institute and sequentially enrolled onto the study at 37 weeks of age. All dogs were eligible for the study as there were no exclusion criteria. There were 15 entire females and 13 neutered males. The dog's body weight at 37 weeks of age ranged from 3.92 kg to 16.28 kg (average 11.69 kg).

The dogs were fed a commercial dry diet (Royal Canin® Yorkshire terrier 29 Junior) from weaning up to 14 weeks of age. At 14 weeks of age 6 dogs remained on this diet, 6 were weaned onto a commercial wet diet (Cesar® puppy with chicken & rice with a carrot topping) and the remaining 16 dogs were fed a simultaneous offering of the dry and wet diets. The diet groups were randomized within litter and balanced by gender.

This study was approved by Waltham Animal Welfare and Ethical Review Body (AWERB) and run under licensed authority in accordance with the UK Animals (Scientific Procedures) Act 1986. The suitability of dogs for the study was determined by a veterinarian, based on a physical examination and a review of the dogs' veterinary history. The dogs were housed in environmentally enriched kennels which included indoor and outdoor access. All the dogs were provided with a comprehensive socialization and training program which was adjusted to their individual needs.

### Clinical Measures

As part of the larger study, the dogs had their oral health determined at 37 weeks of age and then re-assessed at eight-week (+/−1 week) intervals up to a maximum of 61 weeks of age (Wallis et al., 2019). Clinical assessments were performed under general anesthesia during which the levels of gingivitis and periodontitis were assessed around the whole gingival margin of every tooth. Dogs were examined prior to each general anesthesia and were removed from the study once 12 or more teeth developed early signs of periodontitis.

### Sample Collection

SG (n=43) and GM (n=43) plaque samples were collected when dogs were under general anesthesia for assessment of periodontal health: 56 samples at 37 weeks of age; 26 samples at 45 weeks of age; and 4 samples at 53 weeks of age. GM plaque was collected prior to scoring gingivitis. Collection involved sweeping a sterile periodontal probe across the crown of the tooth just above the gingival margin. SG plaque samples were collected whilst scoring gingivitis. This involved placing a sterile periodontal probe under the gingival margin and sweeping it along the base of the crown of the tooth.

In both instances, the probe was placed into a 0.5 ml Eppendorf tube containing 300 μl TE buffer (10 mM Tris-HCL, 1 mM disodium EDTA, pH8.0; Sigma-Aldrich) and agitated to remove the plaque. GM plaque was collected from every tooth in the mouth and placed into the same Eppendorf tube. Likewise, SG plaque was collected from every tooth in the mouth and placed into another Eppendorf tube. This resulted in two plaque samples for each dog on each sampling occasion. The plaque samples were stored on dry ice for a maximum of 30 minutes prior to storage at −80° C.

### DNA Extraction

DNA was extracted using the Masterpure™ Gram positive DNA purification kit (Epicentre, #MGP04100). The manufacturer's instructions were followed but with an additional overnight lysis. After centrifugation of the plaque samples at 5000×g for 10 minutes, the cell pellet was resuspended in 150 μl of TE buffer and 1 μl Ready-Lyse™ Lysozyme Solution added. The lysis mix was incubated at 37° C. for 18 hours overnight. Following DNA extraction, the DNA pellet was suspended in TE buffer.

Sequencing of the 16S rRNA Gene

The 16S rRNA gene, variable regions 3 & 4, was amplified using the Extensor Hi-Fidelity PCR Enzyme Mix (AB-0792, Thermo, UK) and universal bacterial primers 319F and 806R. Each primer contains a linker sequence, index sequence and heterogeneity spacer (Fadrosh et al., 2014). The PCR mixture contained 25 μl Phusion® High-Fidelity PCR Master Mix with HF Buffer (MO531, New England Biolabs, UK), 5 μl of each primer (1 μM), 10 μl template DNA, 3.5 μl nuclease free water and 1.5 μl DMSO, prepared in a 96-well format. The PCR cycling conditions consisted of an initial denaturation step at 98° C. (30 s), followed by 30 cycles of 98° C. (15 s), 58° C. (15 s) and 72° C. (15 s) and a final elongation at 72° C. (60 s). Library preparation and sequencing was carried out by Eurofins Genomics, Germany. In brief, the 16S amplicons were quantified using the Quant-iT™ PicoGreen® dsDNA Assay Kit (Invitrogen, UK) and then pooled in equimolar amounts. The 16S rRNA amplicon libraries were sequenced on a MiSeq (Illumina) using v3 chemistry and bi-directional 300 base pair mode.

### Processing of Sequence Data

Forward and reverse reads were assembled into contiguous sequences spanning the entire V3-V4 regions using FLASH assembler (Magoc and Salzberg, 2011). Linker sequences were removed using TagCleaner (Schmieder et al., 2010) and sequences de-multiplexed in QIIME using split_libraries_fastq.py. Chimeric sequences were removed using userarch6 (Edgar, 2010). Sequences were clustered at ≥98% identity using uclust (Caporaso et al., 2010) to generate operational taxonomic units (OTUs). The most abundant sequence in each OTU were selected as the representative. The representative sequences were annotated using blastall 2.2.25 (Altschul et al., 1990) and the Silva databases. The Silva database contains full-length 16S rRNA sequences to previously identified canine oral taxa (COT) and feline oral taxa (FOT) (Pruesse et al., 2007). These were deposited in GenBank and received accession numbers JN713151-JN713566 and KM461942-KM462187 (Dewhirst et al., 2012; Dewhirst et al., 2015). OTUs with an average proportion in both diet groups of ≤0.01%, or present in less than two samples, were combined and termed “rare”.

### Statistical Analysis

Statistical analyses were performed using R version 4.0. The primary measures for oral health status of each dog were mean gingivitis score (average of four measurements for each tooth and then average of all teeth in the mouth), proportion of healthy teeth in the mouth and the proportion of teeth with periodontitis in the mouth. The primary microbiota measure was operational taxonomic units (OTUs). A non-metric multidimensional scaling (nMDS) was performed using a Bray Curtis distance matrix calculated from the sequence count for the 187 non-rare OTUs to determine whether there was separation of individuals by diet or sample type.

To investigate changes in OTUs associated with diet group, a factor analysis was performed, followed by a linear mixed effect model to determine factor significance. Factor analysis is a statistical method used to describe variability among observed, correlated variables in a lower number of unobserved variables called factors. The log transformed relative abundances (+2 to the count and +4 to the total) were explored for the optimal number of factors, and then fitted to factor analyses using this optimal value. A linear mixed effect model was then applied to each factor. The scores for that factor were the response variable with diet group (wet, dry or mixed), sample type (GM or SG) and their interaction as fixed effects and individual (dog) as the random effect. The p-value for the differences between diet groups within each sample type, and the differences between each sample type within each diet group were then reported.

The Shannon diversity index was calculated for each sample using all OTUs i.e., including those that were classified as rare. A linear mixed effect model was applied with total sequence counts as a covariate and Shannon diversity index as the response and diet group, sample type and their interaction as fixed effects and dog as a random effect. A Tukey post-hoc test was applied to compare each combination of sample type and diet to each other.

### Results

### Sequence Quality

Sequencing of 86 samples using the Illumina MiSeq platform resulted in the generation of 7,141,323 paired end reads. Following removal of rare sequences, 6,725,682 paired-end reads remained (94.2%) with an average number of reads per sample of 83,039 (+/−43,277).

Clustering of sequence reads at ≥98% identity resulted in the generation of 172 OTUs following filtering of those lower than 0.05% or that were not present in two or more samples. Of these, 165 (95.9%) had a sequence identity of ≥98% to sequences within the Silva database and the remaining 7 OTUs (4.1%) had identities to sequences within the database ranging from 96.06% to 97.79%. A total of 118 OTUs (68.6%) mapped to sequences previously isolated from plaque samples from dogs and cats. With respect to the other OTUs, 28 (16.3%) could be assigned taxonomy at the species level, 19 (11.1%) to genus, 5 (2.9%) to family, 1 (0.6%) to class and 1 (0.6%) to order.

### Taxonomy

In total, 12 phyla, 22 classes, 36 orders, 58 families, 86 genera and 163 species were identified within the canine SG and GM plaque samples. The most abundant phyla were Firmicutes (34.9%), Proteobacteria (19.0%), Bacteroidetes (18.0%), Actinobacteria (10.2%) and Fusobacteria (6.1%). The remaining 7 phyla comprised Patescibacteria (4.3%), Spirochaetes (3.3%), Epsilonbacteraeota (2.3%), Synergistetes (0.9%), SR1 (0.5%), Chlorobi (0.4%) and Tenericutes (0.2%).

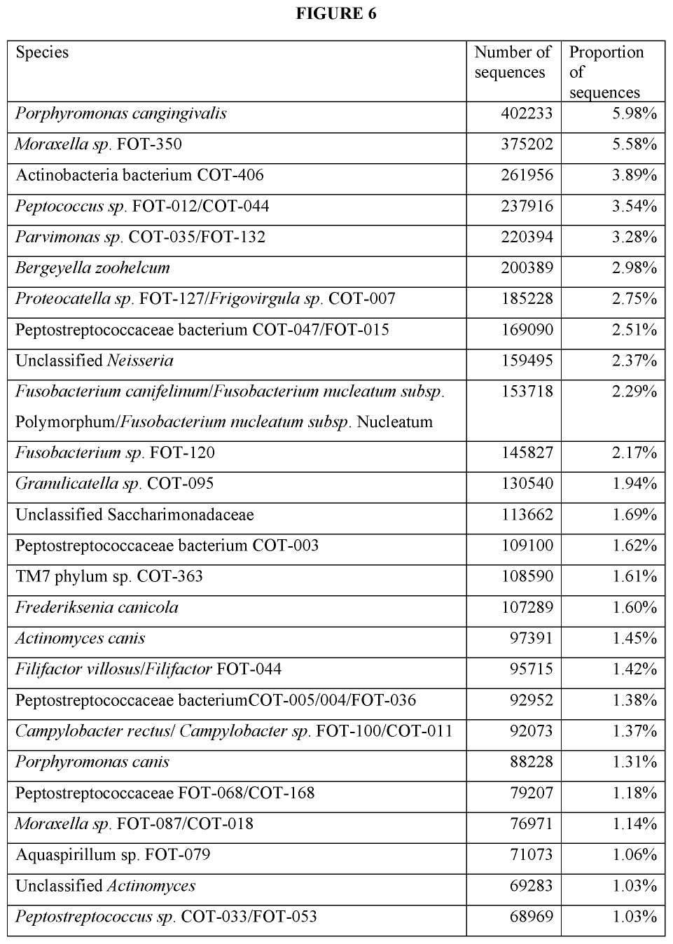

There were 26 species that were present at a relative abundance >1.0% and these together accounted for 58.2% of total sequence reads (FIG. 6). The Canine Oral Taxon numbers (COT) and Feline Oral Taxon numbers (FOT) are provided for the species listed if available (Dewhirst et al., 2012; Dewhirst et al., 2015). The species with the highest relative abundance in canine plaque was Porphyromonas cangingivalis representing 6% of the total sequence reads.

### Changes in Microbiota

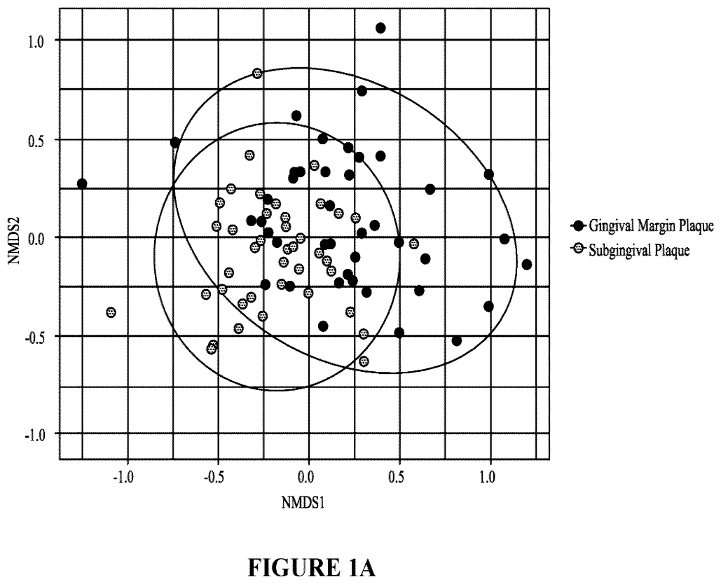

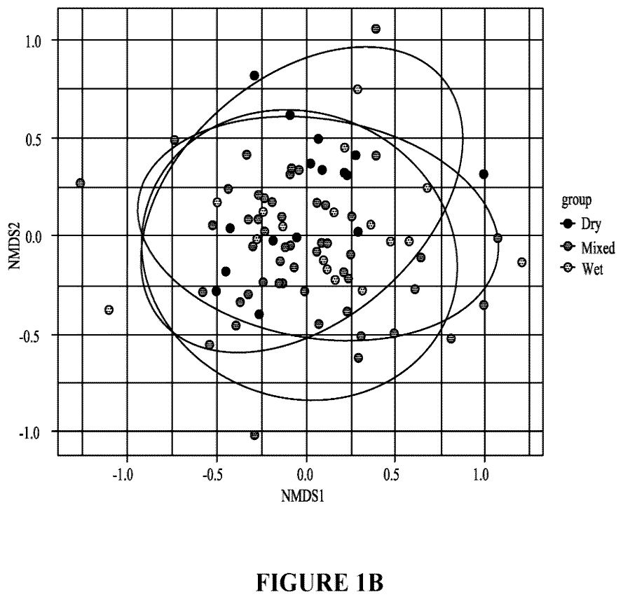

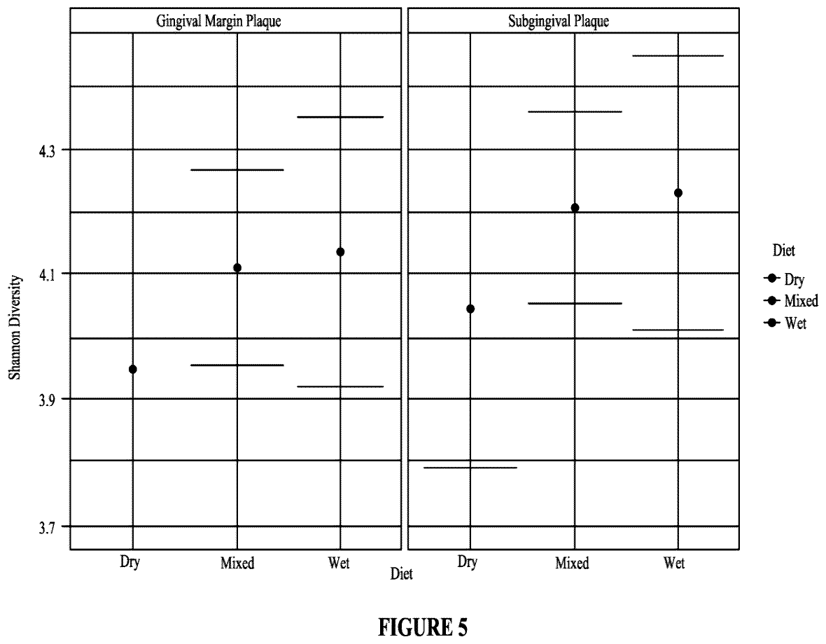

The Shannon diversity did not significantly differ between SG and GM plaque when dogs were fed commercial dry, commercial wet or a mixture of the two diets (all pairwise comparisons P<0.05; FIG. 5). This finding demonstrates minimal differences in the diversity of species across diet types or plaque types. There was also no clear separation of samples by diet group following exploratory multivariate analyses (nMDS), demonstrating that diet also had little influence on the overall microbiota composition of SG and GM plaque (FIG. 1A). However, there was evidence of separation between GM and SG sample types (FIG. 1B), showing the two locations have differing bacterial communities.

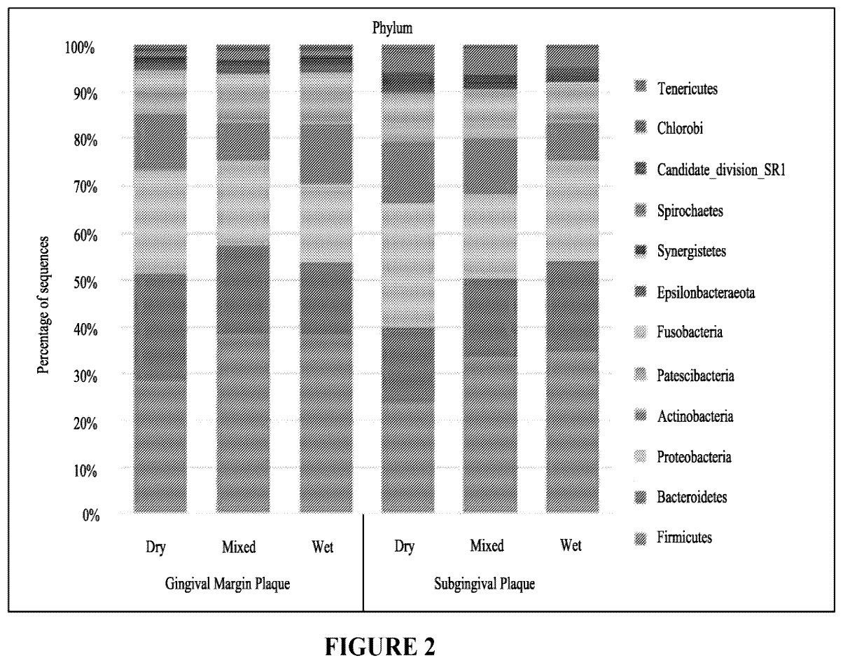

Visual inspection of the phylum level data showed differences in relative abundance by sample type and diet group (FIG. 2). For example, dogs fed the dry diet had a lower relative abundance of Firmicutes in both GM and SG plaque. In SG plaque, this was associated with a higher relative abundance of Proteobacteria whereas, in GM plaque it was linked with a higher relative abundance of Proteobacteria and Bacteroidetes.

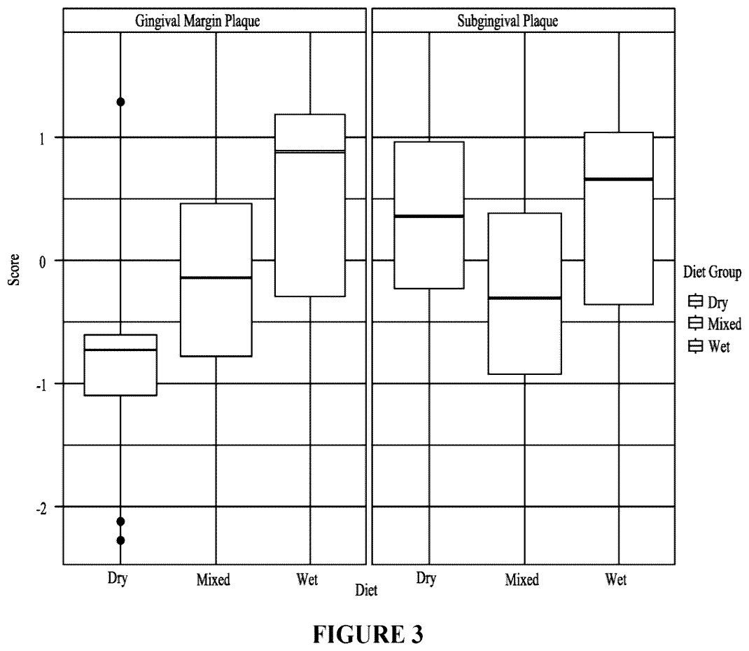

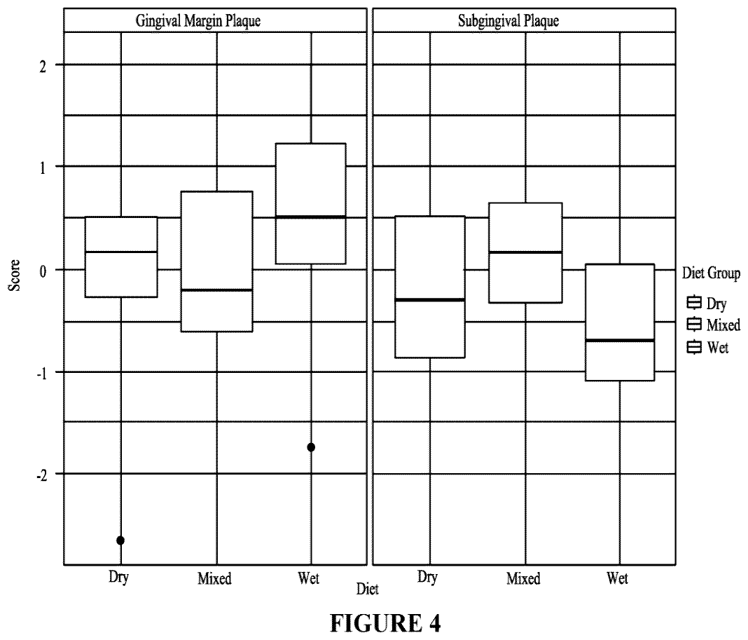

Exploratory Factor Analysis was used to determine if any underlying structure existed within the OTUs identified within the GM and SG plaque samples. This addresses how bacterial groups change across sample types while also examining the interactions between bacterial groups, with a total of eight factors identified. Confirmatory factor analysis determined that there were significant relationships between the patterns in the OTU data and diet and sample type. Factor group MR3 indicated significant differences in the OTUs (weighted average) between wet and dry commercial diets in GM plaque (P=0.019; FIG. 3). Factor group MR7 indicated a statistically significant difference between GM and SG plaque in dogs fed a wet diet (P=0.043; FIG. 4).

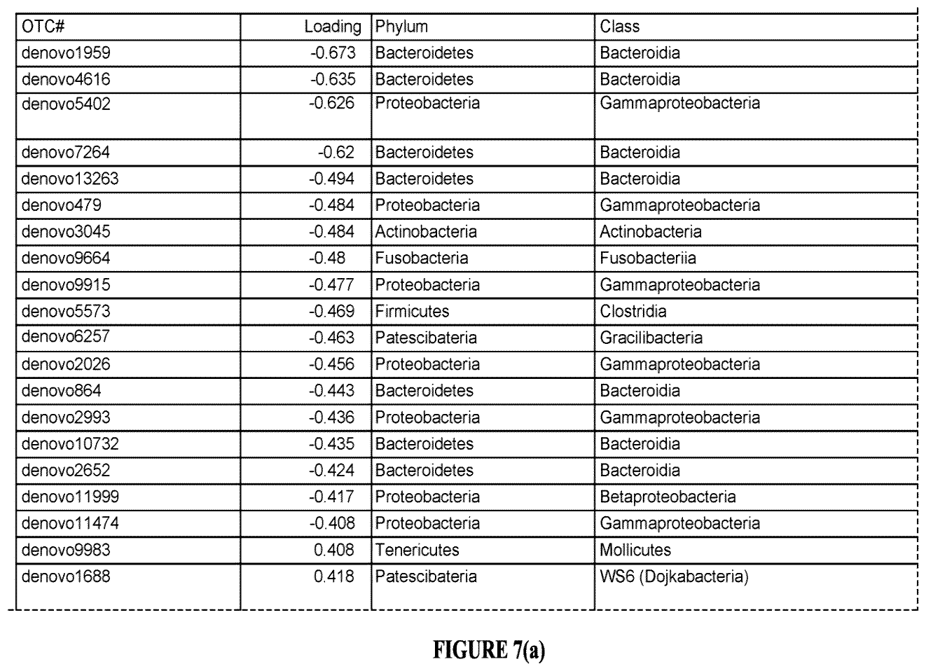

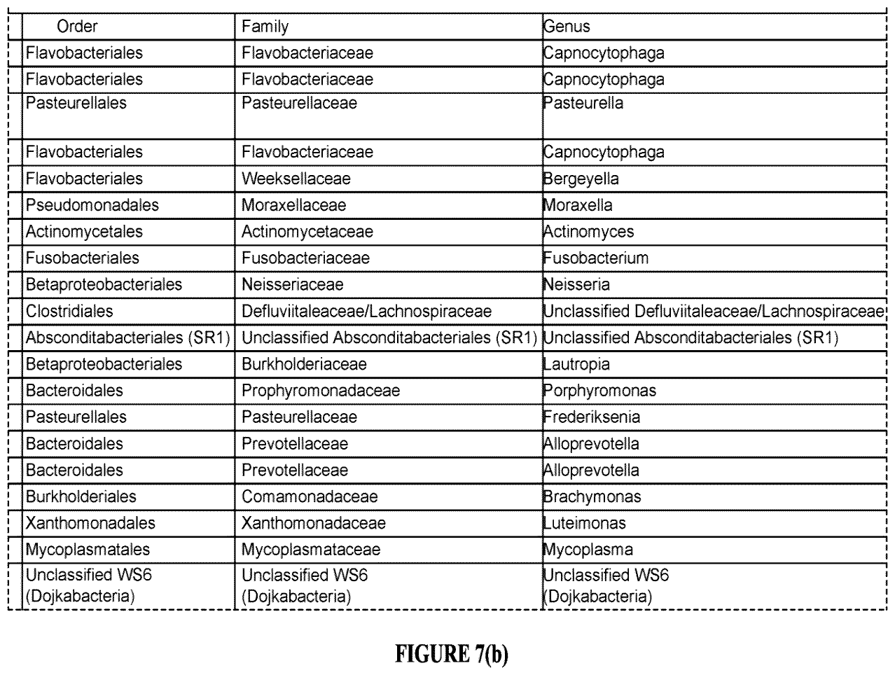

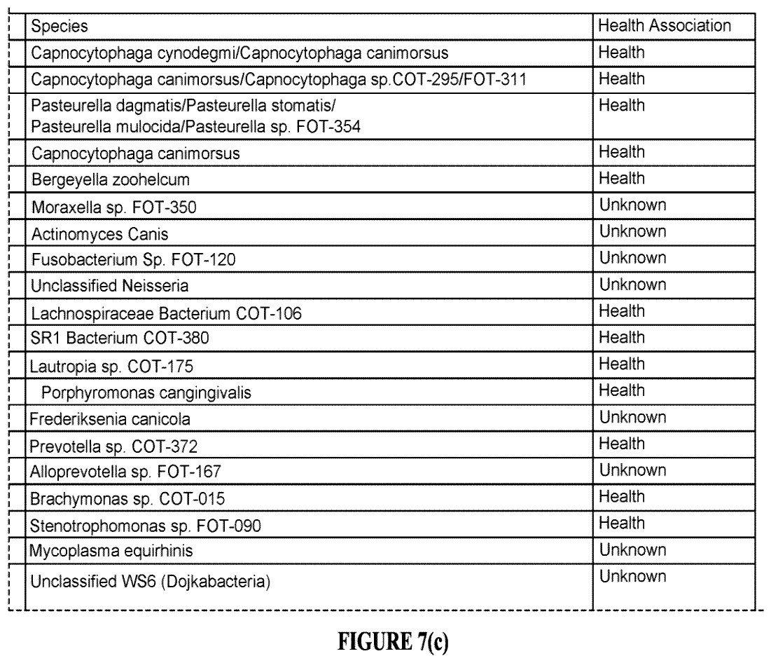

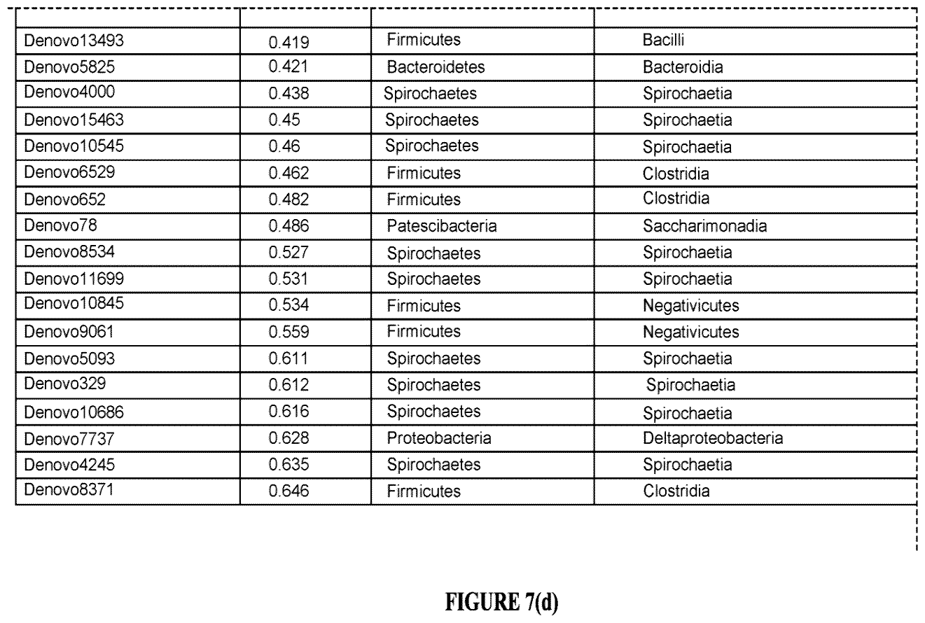

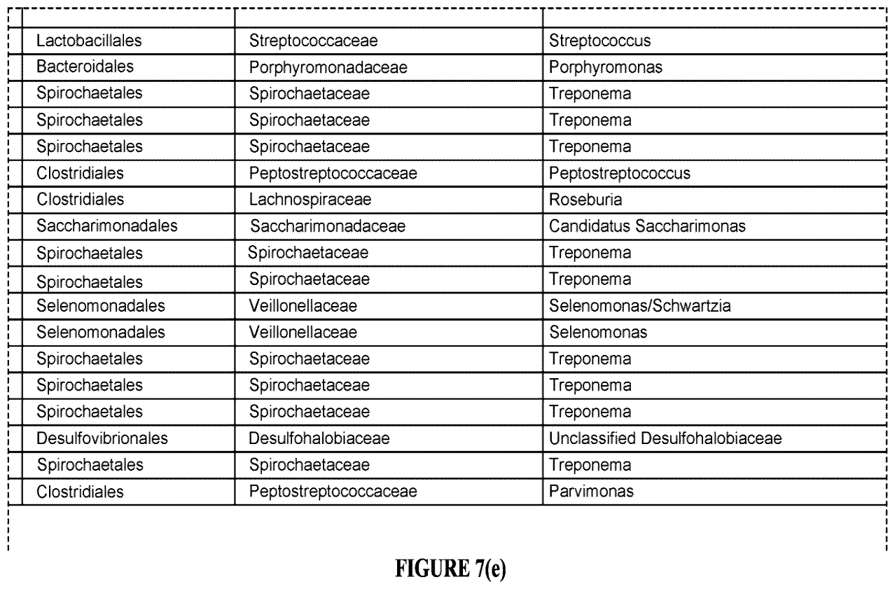

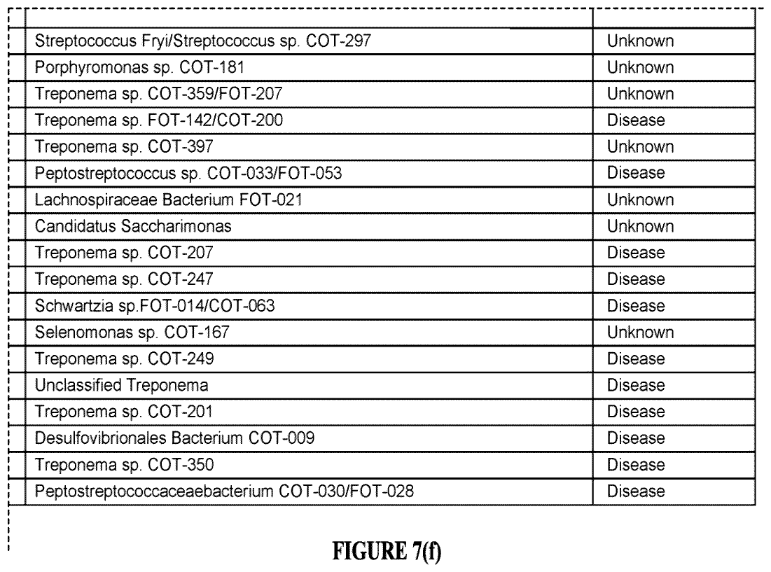

The OTUs that were most descriptive of factor group MR3 (loadings value >0.5 or <0.5) and are associated with changes in microbiota composition between wet and dry commercial diets in GM plaque are summarized in FIGS. 7A-7F. GM plaque from dogs fed the wet diet were associated with OTUs with positive loadings. In contrast, GM plaque from dogs fed the dry diet were associated with OTUs with negative loadings. The dogs fed a mixture of wet and dry diets had a mixture of OTUs with positive and negative loadings. In general, the OTUs with positive loadings (i.e. the wet diet) have previously been shown to be associated with periodontal disease and those with negative loadings (i.e. the dry diet) have previously been shown to be associated with healthy gingiva (Davis et al., 2013). OTUs with positive loadings were mostly members of the phylum Firmicutes (e.g., species belonging to the genus Schwartzia and Selomonas and the family Peptostreptococcaceae) and Spirochaetota/Spirochaetes (e.g., species belonging to the genus Treponema). OTUs with negative loadings belonged to the phylum Bacteroidetes (e.g., species belonging to the genus Capnocytophaga) and Proteobacteria (e.g., species from the genus Pasteurella).

### Periodontal Health Status

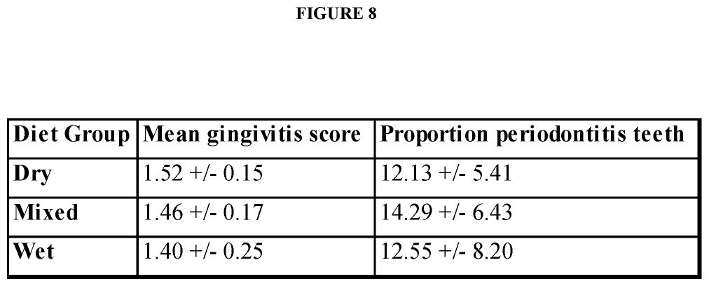

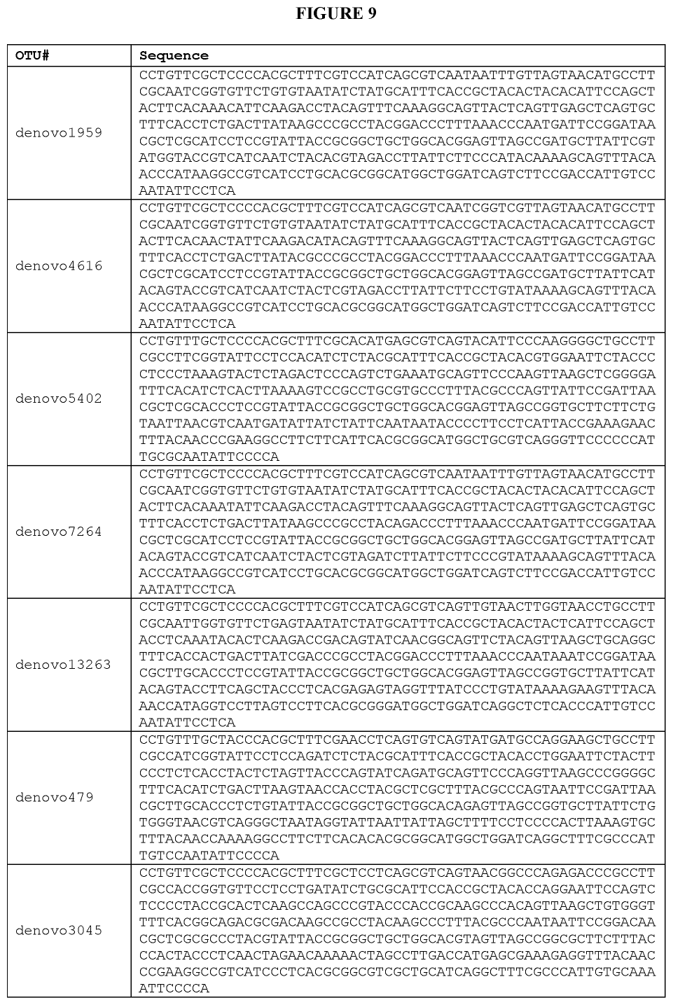

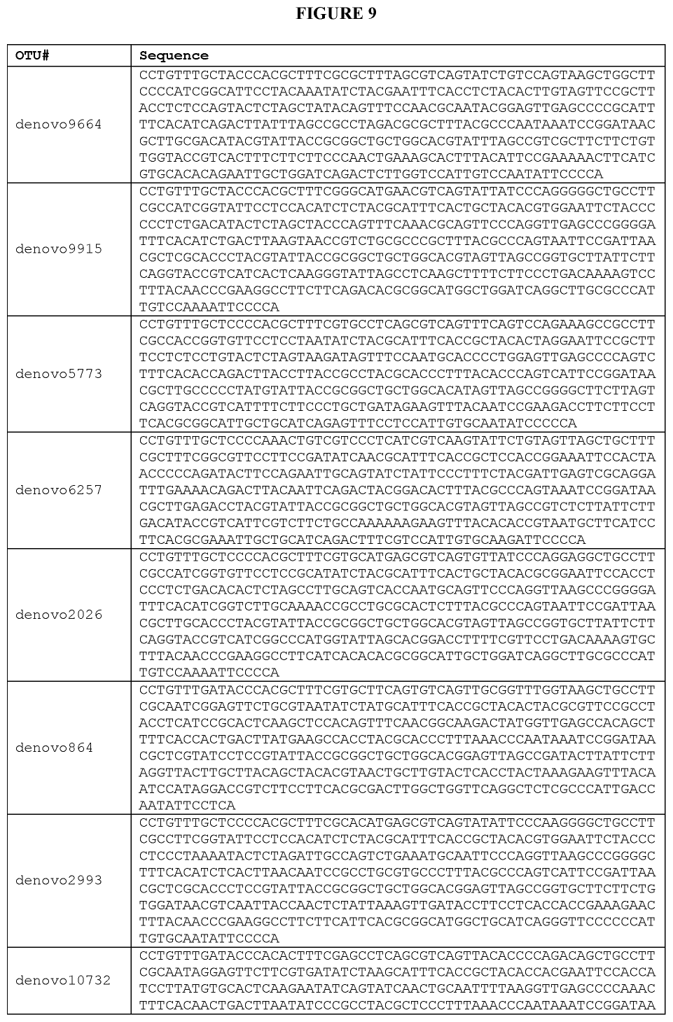

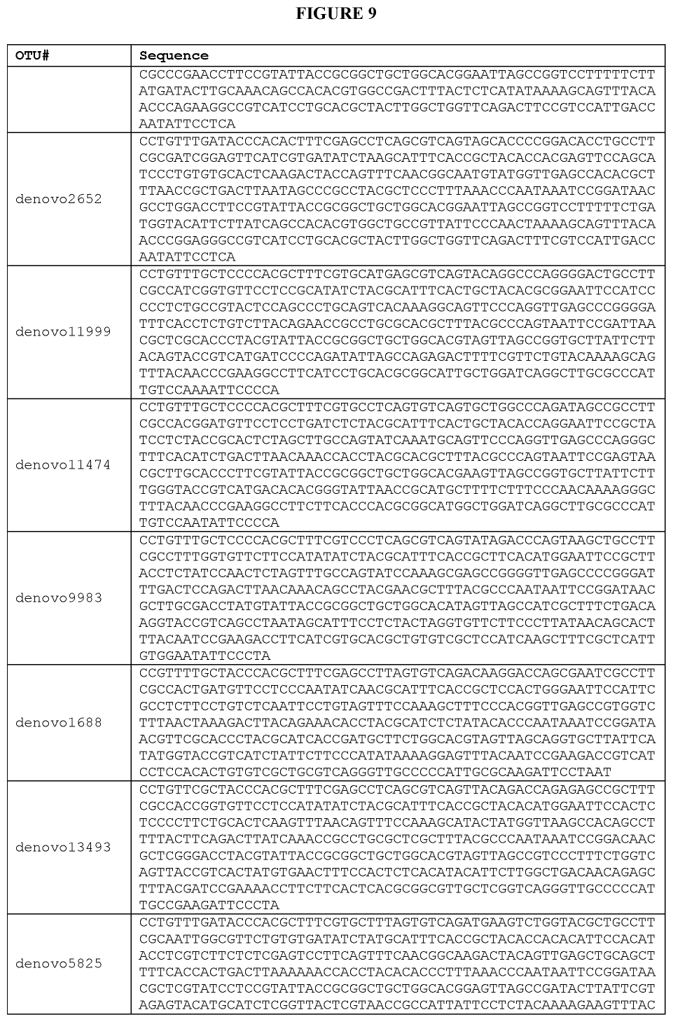

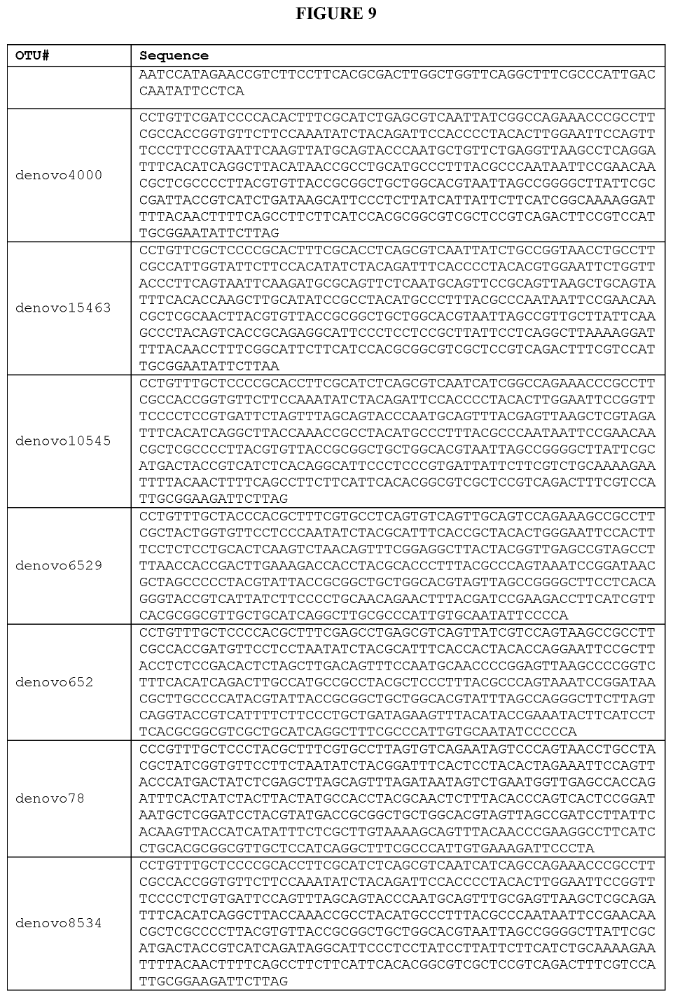

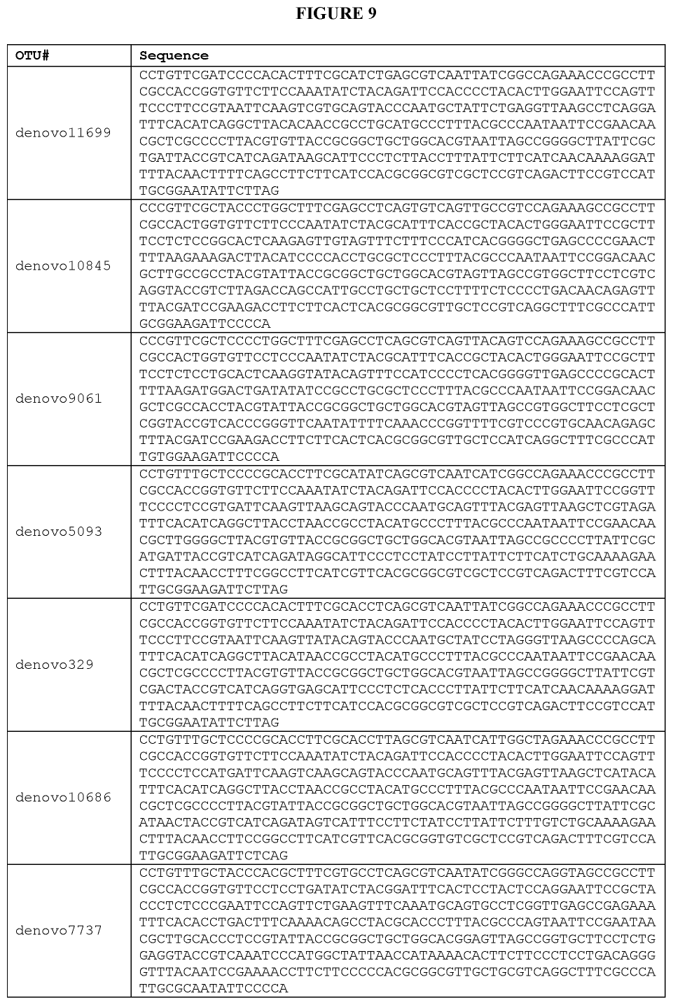

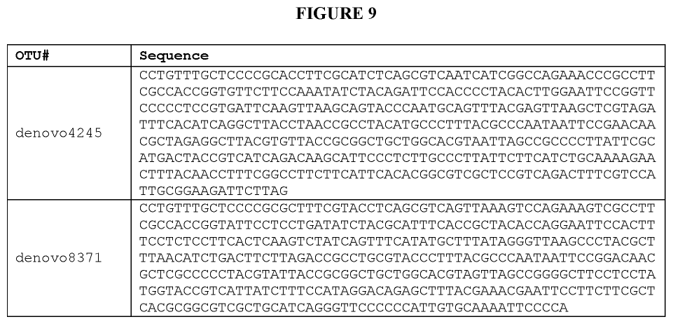

The clinical health status was similar across diet groups with the average gingivitis score ranging from 1.40 to 1.52 and the proportion of periodontitis teeth ranging from 12.13 to 14.29 (FIG. 8). This data demonstrated that the consistency of the diet did not differentially alter the periodontal health status of this population of Yorkshire terriers over the first year of their life.

### Discussion

This evaluation highlighted the impact of commercial diet types on the bacterial composition of canine dental plaque. GM and SG plaque samples from Yorkshire terriers fed a commercial dry diet had a lower relative abundance of Firmicutes when compared to those fed a wet commercial diet. This finding was associated with a higher relative abundance of Proteobacteria (SG & GM) and Bacteroidetes (GM only). A number of bacterial species belonging to the phylum Firmicutes have been shown to be associated with early periodontal disease in dogs whereas many of the bacterial species from the phyla Bacteroidetes and Proteobacteria have been shown to be associated with periodontal health (Davis et al., 2013; Wallis et al., 2015; Wallis et al., 2021). A factor analysis supported these visual observations and provided evidence that diet had no effect on SG plaque.

Although the impact of wet and dry diets on plaque and calculus accumulation is not reported here, the data from the current study of Yorkshire terriers supports that finding that dry diets help modulate, reduce or manage the development of mature plaque and consequently colonization of the tooth surface by bacterial species that have been associated with periodontal disease. In this study of Yorkshire terriers aged 37 to 61 weeks, diet had little effect on their overall periodontal health status (gingivitis and proportion of teeth with periodontitis). This is in partial agreement with a study that found that dogs fed a dry food had less gingivitis but little effect on tooth mobility, tooth loss and periodontitis (Golden et al., 1982). Similarly, a study of 1,350 dogs did not show an association between the levels of gingival inflammation and periodontal bone loss when dogs fed dry food were compared to those fed anything other than dry food only unless chewing materials were also provided (Harvey et al., 1996). This is in contrast to other studies that suggest soft diets are associated with an increased incidence and severity of periodontal disease compared to predominantly dry diets (Gawor et al., 2006; Watson, 1994; Buckley et al., 2011). Despite the fact Yorkshire terriers are prone to developing periodontal disease from a young age (Wallis et al., 2019), if diets had been fed for a longer period of time, a greater difference in their periodontal health status can emerged.

Multivariate analysis indicated that the microbial composition of GM and SG plaque differed. This was confirmed by the exploratory factor analysis which identified a significant relationship between a number of OTUs and sample type in dogs fed a wet diet. The GM plaque samples from dogs fed the commercial wet diet correlated with OTUs with positive loadings and these tended to be bacterial species that prefer aerobic conditions. In contrast, the SG plaque samples were correlated with OTUs with negative loadings, and these tended to be bacterial species that favor anaerobic conditions. This concurs with previous studies where SG plaque samples have been shown to have a significantly lower proportion of aerobic taxa and significantly higher proportion of anaerobic taxa than GM samples (Ruparell et al., 2017).

The commercial dry diet reduced the build-up of mature plaque on the surface of dogs' teeth compared to wet commercial diets. This finding in addition to other methods to maintain dental hygiene should be considered to ensure effective management of periodontal disease in dogs.

### REFERENCES

Adler, C. J., Malik, R., Browne, G. V, Norris, J. M., 2016. Diet may influence the oral microbiome composition in cats. Microbiome 4, 23. https://doi.org/10.1186/s40168-016-0169-y
Altschul, S. F., Gish, W., Miller, W., Myers, E. W., Lipman, D. J., 1990. Basic local alignment search tool. J. Mol. Biol. 215, 403-410. https://doi.org/10.1016/s0022-2836 (05) 80360-2
Anon, 1985. Survey on the health of pet animals, 2nd report. Japan Small Anim. Vet. Assoc. 25.
Boyce, E. N., Logan, E. I., 1994. Oral health assessment in dogs: study design and results. J Vet Dent 11, 64-70.
Buckley, C., Colyer, A., Skrzywanek, M., Jodkowska, K., Kurski, G., Gawor, J., Ceregrzyn, M., 2011. The impact of home-prepared diets and home oral hygiene on oral health in cats and dogs. Br J Nutr 106 Suppl, S124-127. https://doi.org/10.1017/S0007114511000821
Butković, V., Šimpraga, M., Šehić, M., Stanin, D., Sušić, V., Capak, D., Kos, J., 2001. Dental diseases of dogs: a retrospective study of radiological data. Acta Vet. Brno 70, 203-208.
Caporaso, J. G., Bittinger, K., Bushman, F. D., DeSantis, T. Z., Andersen, G. L., Knight, R., 2010. PyNAST: a flexible tool for aligning sequences to a template alignment. Bioinformatics 26, 266-267. https://doi.org/10.1093/bioinformatics/btp636
Davis, I. J., Wallis, C., Deusch, O., Colyer, A., Milella, L., Loman, N., Harris, S., 2013. A cross-sectional survey of bacterial species in plaque from client owned dogs with healthy gingiva, gingivitis or mild periodontitis. PLOS One 8, e83158. https://doi.org/10.1371/journal.pone.0083158
DeBowes, L. J., Mosier, D., Logan, E., Harvey, C. E., Lowry, S., Richardson, D. C., 1996. Association of periodontal disease and histologic lesions in multiple organs from 45 dogs. J Vet Dent 13, 57-60.
Dewhirst, F. E., Klein, E. A., Bennett, M. L., Croft, J. M., Harris, S. J., Marshall-Jones, Z. V, 2015. The feline oral microbiome: a provisional 16S rRNA gene based taxonomy with full-length reference sequences. Vet Microbiol 175, 294-303. https://doi.org/10.1016/j.vetmic.2014.11.019
Dewhirst, F. E., Klein, E. A., Thompson, E. C., Blanton, J. M., Chen, T., Milella, L., Buckley, C. M., Davis, I. J., Bennett, M. L., Marshall-Jones, Z. V, 2012. The canine oral microbiome. PLOS One 7, e36067. https://doi.org/10.1371/journal.pone.0036067
Edgar, R. C., 2010. Search and clustering orders of magnitude faster than BLAST. Bioinformatics 26, 2460-2461. https://doi.org/10.1093/bioinformatics/btq461
Fadrosh, D. W., Ma, B., Gajer, P., Sengamalay, N., Ott, S., Brotman, R. M., Ravel, J., 2014. An improved dual-indexing approach for multiplexed 16S rRNA gene sequencing on the Illumina MiSeq platform. Microbiome 2, 6. https://doi.org/10.1186/2049-2618-2-6
Gawor, J. P., Reiter, A. M., Jodkowska, K., Kurski, G., Wojtacki, M. P., Kurek, A., 2006. Influence of diet on oral health in cats and dogs. J Nutr 136, 2021S-2023S.
Glickman, L. T., Glickman, N. W., Moore, G. E., Goldstein, G. S., Lewis, H. B., 2009. Evaluation of the risk of endocarditis and other cardiovascular events on the basis of the severity of periodontal disease in dogs. J Am Vet Med Assoc 234, 486-494. https://doi.org/10.2460/javma.234.4.486
Golden, A. L., Stoller, N., Harvey, C. E., 1982. A survey of oral and dental diseases in dogs anesthetized at a veterinary hospital. J. Am. Anim. Hosp. Assoc. 18, 891.
Gorrel, C., 2013. Veterinary Dentistry for the General Practitioner. Elsevier Saunders Ltd. https://doi.org/https://doi.org/10.1016/C2011-0-05461-3
Harvey, C. E., 2005. Management of periodontal disease: understanding the options. Vet Clin North Am Small Anim Pr. 35, 819-836. https://doi.org/10.1016/j.cvsm.2005.03.002
Harvey, C. E., 1998. Periodontal disease in dogs. Etiopathogenesis, prevalence, and significance. Vet Clin North Am Small Anim Pr. 28, 1111-1128.
Harvey, C. E., Shofer, F. S., Laster, L., 1996. Correlation of diet, other chewing activities and periodontal disease in North American client-owned dogs. J Vet Dent 13, 101-105.
Harvey, C. E., Shofer, F. S., Laster, L., 1994. Association of age and body weight with periodontal disease in North American dogs. J Vet Dent 11, 94-105.
Hoffman, T., Gaengler, P., 1996. Epidemiology of periodontal disease in poodles. J. Small Anim. Pract. 37, 309-316.
Isogai, H., Isogai, E., Okamoto, H., Shirakawa, H., Nakamura, F., Matsumoto, T., Watanabe, T., Miura, H., Aoi, Y., Kagota, W., Takano, K., 1989. Epidemiological Study on Periodontal Diseases and some Other Dental Disorders in Dogs. Japanese J. Vet. Sci. 51, 1151-1162.
Kortegaard, H. E., Eriksen, T., Baelum, V., 2008. Periodontal disease in research beagle dogs—an epidemiological study. J Small Anim Pr. 49, 610-616. https://doi.org/10.1111/j.1748-5827.2008.00609.x
Kyllar, M., Witter, K., 2005. Prevalence of dental disorders in pet dogs. Vet. Med. (Praha). 50, 496-505.
Lindhe, J., Hamp, S. E., Loe, H., 1975. Plaque induced periodontal disease in beagle dogs. A 4-year clinical, roentgenographical and histometrical study. J Periodontal Res 10, 243-255.
Logan, E. I., 2006. Dietary influences on periodontal health in dogs and cats. Vet Clin North Am Small Anim Pr. 36, 1385-401, ix. https://doi.org/10.1016/j.cvsm.2006.09.002
Lund, E. M., Armstrong, P. J., Kirk, C. A., Kolar, L. M., Klausner, J. S., 1999. Health status and population characteristics of dogs and cats examined at private veterinary practices in the United States. J Am Vet Med Assoc 214, 1336-1341.
Magoc, T., Salzberg, S. L., 2011. FLASH: fast length adjustment of short reads to improve genome assemblies. Bioinformatics 27, 2957-2963. https://doi.org/10.1093/bioinformatics/btr507
Niemiec, B., 2012. Veterinary Periodontology. Wiley.
Niemiec, B. A., 2008. Oral pathology. Top Companion Anim Med 23, 59-71. https://doi.org/10.1053/j.tcam.2008.02.002
O'Neill, D. G., Church, D. B., McGreevy, P. D., Thomson, P. C., Brodbelt, D. C., 2014. Prevalence of disorders recorded in dogs attending primary-care veterinary practices in England. PLOS One 9, e90501. https://doi.org/10.1371/journal.pone.0090501
Oba, P. M., Carroll, M. Q., Alexander, C., Somrak, A. J., Keating, S. C. J., Sage, A. M., Swanson, K. S., 2021. Dental Chews Positively Shift the Oral Microbiota of Adult Dogs. J. Anim. Sci. https://doi.org/10.1093/jas/skab100
Pavlica, Z., Petelin, M., Juntes, P., Erzen, D., Crossley, D. A., Skaleric, U., 2008. Periodontal disease burden and pathological changes in organs of dogs. J Vet Dent 25, 97-105.
Pereira Dos Santos, J. D., Cunha, E., Nunes, T., Tavares, L., Oliveira, M., 2019. Relation between periodontal disease and systemic diseases in dogs. Res Vet Sci 125, 136-140. https://doi.org/10.1016/j.rvsc.2019.06.007
Pruesse, E., Quast, C., Knittel, K., Fuchs, B. M., Ludwig, W., Peplies, J., Glockner, F. O., 2007. SILVA: a comprehensive online resource for quality checked and aligned ribosomal RNA sequence data compatible with ARB. Nucleic Acids Res 35, 7188-7196. https://doi.org/10.1093/nar/gkm864
Robinson, N. J., Dean, R. S., Cobb, M., Brennan, M. L., 2016. Factors influencing common diagnoses made during first-opinion small-animal consultations in the United Kingdom. Prev Vet Med 131, 87-94. https://doi.org/10.1016/j.prevetmed.2016.07.014
Ruparell, A., Wallis, C., Holcombe, L. J., Davis, I., 2017. A comparative analysis of the microbiota of subgingival and gingival margin plaque across dog's with healthy gingiva, gingivitis and early periodontitis. WALTHAM internal report WCR4966.
Ruparell, A., Warren, M., Staunton, R., Deusch, R., Dobenecker, B., Wallis, C., O'Flynn, C., McGenity, P., Holcombe, L., 2019. Effect of feeding a daily oral care chew on the composition of plaque microbiota in dogs. Prep.
Salt, C., Morris, P. J., German, A. J., Wilson, D., Lund, E. M., Cole, T. J., and Butterwick, R. F., 2017. Growth standard charts for monitoring bodyweight in dogs of different sizes. PLOS One 12, e0182064. https://doi.org/10.1371/journal.pone.0182064
Schmieder, R., Lim, Y. W., Rohwer, F., Edwards, R., 2010. TagCleaner: Identification and removal of tag sequences from genomic and metagenomic datasets. BMC Bioinformatics 11, 341. https://doi.org/10.1186/1471-2105-11-341
Wallis, C., Holcombe, L. J., 2020. A review of the frequency and impact of periodontal disease in dogs. J Small Anim Pr. 61, 529-540. https://doi.org/10.1111/jsap.13218
Wallis, C., Marshall, M., Colyer, A., O'Flynn, C., Deusch, O., Harris, S., 2015. A longitudinal assessment of changes in bacterial community composition associated with the development of periodontal disease in dogs. Vet. Microbiol. 181, 271-282. https://doi.org/10.1016/j.vetmic.2015.09.003
Wallis, C., Milella, L., Colyer, A., O'Flynn, C., Harris, S., Holcombe, L. J., 2021. Subgingival microbiota of dogs with healthy gingiva or early periodontal disease from different geographical locations. BMC Vet. Res. 17, 1-19. https://doi.org/10.1186/s12917-020-02660-5
Wallis, C., Pesci, I., Colyer, A., Milella, L., Southerden, P., Holcombe, L. J., Desforges, N., 2019. A longitudinal assessment of periodontal disease in Yorkshire terriers. BMC Vet Res 15, 207. https://doi.org/10.1186/s12917-019-1923-8
Watson, A. D., 1994. Diet and periodontal disease in dogs and cats. Aust Vet J 71, 313-318.

Although the presently disclosed subject matter and its advantages have been described in detail, it should be understood that various changes, substitutions, and alterations can be made herein without departing from the spirit and scope of the disclosed subject matter as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A method of modulating the oral microbiota in a companion animal, comprising feeding the animal a dry pet food.

2. The method of claim 1, wherein modulating the oral microbiota results in an increase in bacteria associated with good oral health.

3. The method of claim 2, wherein modulating the oral microbiota results in an increase in at least one bacterial taxa species selected from the group consisting of Capnocytophaga cynodegmi, Capnocytophaga canimorsus, Capnocytophaga sp. COT-295/FOT-311, Pasteurella dagmatis, Pasteurella stomatis, Pasteurella mulocida, Pasteurella sp. FOT-354, Bergeyella zoohelcum, Lachnospiraceae bacterium COT-106, SR1 bacterium COT-380, Lautropia sp. COT-175, Porphyromonas cangingivalis, Prevotella sp. COT-372, Brachymonas sp. COT-015, Stenotrophomonas sp. FOT-090, and a combination thereof.

4. A method of improving the oral health of an animal by modulating the animal's oral microbiota, comprising feeding the animal a dry pet food.

5. A method of decreasing the formation of a disease-associated bacterial population on a biological surface of a companion animal in need thereof, comprising feeding the animal a dry pet food.

6. The method of claim 5, wherein the biological surface is an oral surface.

7. The method of claim 6, wherein the oral surface is a subgingival dental surface, a gingival margin dental surface, a supragingival dental surface from the cheek, or a surface of the tongue.

8. The method of claim 6, wherein the oral surface is a gingival margin dental surface, a subgingival dental surface, a supragingival dental surface, or a combination thereof.

9. The method of claim 6, wherein the oral surface is a gingival margin dental surface.

10. The method of claim 6, wherein the oral surface comprises an oral plaque.

11. The method of claim 6, wherein the oral surface comprises at least one bacteria taxa order selected from the group consisting of Spirochaetales, Selenomonadales, Desulfovibrionales, Clostridiales, Fusobacteriales, Bacteroidales, Lactobacillales, Mycoplasmatales, Saccharimonadales, Unclassified WS6 (Dojkabacteria), and a combination thereof.

12. The method of claim 11, wherein the at least one bacteria taxa order is selected from the group consisting of Spirochaetales, Selenomonadales, Desulfovibrionales, Clostridiales, and a combination thereof.

13. The method of claim 6, wherein the oral surface comprises at least one bacteria taxa family selected from the group consisting of Spirochaetaceae, Veillonellaceae, Desulfohalobiaceae, Peptostreptococcaceae, Mycoplasmataceae, Unclassified WS6 (Dojkabacteria), Streptococcaceae, Porphyromonadaceae, Lachnospiraceae, Saccharimonadaceae, and a combination thereof.

14. The method of claim 6, wherein the oral surface comprises at least one bacteria taxa species selected from the group consisting of Treponema sp. COT-207, Treponema sp. COT-247, Schwartzia sp. FOT-014/COT-063, Treponema sp. COT-249, Unclassified Treponema, Treponema sp. COT-201, Treponema sp. FOT-142/COT-200, Desulfovibrionales bacterium COT-009, Treponema sp. COT-350, Peptostreptococcaceae bacterium COT-030/FOT-028, Peptostreptococcaceae sp. COT-033/FOT-053, and a combination thereof.

15. A method of identifying a companion animal at risk of periodontal disease, comprising:
a. obtaining an oral plaque sample from the animal;
b. isolating a nucleic acid from the sample;
c. analyzing the nucleic acid; and
d. identifying at least one bacteria taxa present in the sample;
wherein, if the at least one bacteria taxa is associated with disease, the animal is at risk of periodontal disease.

16. The method of claim 15, wherein the oral plaque sample comprises a subgingival dental plaque, a gingival margin dental plaque, a supragingival dental plaque, a bacterial sample from the cheek, a bacterial sample from the tongue, or a combination thereof.

17. The method of claim 15, wherein the oral plaque sample comprises a gingival margin dental plaque, a subgingival dental plaque, a supragingival dental plaque, or a combination thereof.

18. The method of claim 15, wherein the oral plaque sample is a gingival dental plaque sample.

19. The method of claim 15, wherein the nucleic acid comprises 16S rDNA.

20. The method of claim 15, wherein the at least one bacteria taxa comprise a bacteria taxa order selected from the group consisting of Spirochaetales, Selenomonadales, Desulfovibrionales, Clostridiales, Fusobacteriales, Bacteroidales, Lactobacillales, Mycoplasmatales, Saccharimonadales, Unclassified WS6 (Dojkabacteria), and a combination thereof.

21. The method of claim 15, wherein the at least one bacteria taxa comprise a bacteria taxa family selected from the group consisting of Spirochaetaceae, Veillonellaceae, Desulfohalobiaceae, Peptostreptococcaceae, and a combination thereof.

22. The method of claim 15, wherein the at least one bacteria taxa comprise a bacteria species selected from the group consisting of Treponema sp. COT-207, Treponema sp. COT-247, Schwartzia sp. FOT-014/COT-063, Treponema sp. COT-249, Unclassified Treponema, Treponema sp. COT-201, Treponema sp. FOT-142/COT-200, Desulfovibrionales bacterium COT-009, Treponema sp. COT-350, Peptostreptococcaceae bacterium COT-030/FOT-028, Peptostreptococcaceae sp. COT-033/FOT-053, and a combination thereof.

23. The method of claim 15, further comprising feeding the animal a dry pet food.

24. The method of claim 15, wherein the companion animal is a dog.

25. The method of claim 24, wherein the dog is a toy breed, a small breed, a medium breed, a large breed, or a giant breed.

26. The method of claim 24, wherein the dog is selected from the group consisting of Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy, and Yorkshire Terrier.

27. The method of claim 24, wherein the dog is a Yorkshire Terrier.

28. The method of claim 15, wherein analyzing the DNA comprises sequencing the nucleic acid.


SEQUENCE LISTING (38 sequences)

SEQ ID NO: 1 - DNA, 424 bp, synthetic construct
  misc_feature 1..424: note = Description of sequence: operational taxonomic unit; OUT#: denovo1959
  source 1..424: mol_type = other DNA
cctgttcgctccccacgctttcgtccatcagcgtcaataatttgttagtaacatgccttcgcaatcggtgttctgtgtaatatctatgcatttcaccgctacactacacattccagctacttcacaaacattcaagacctacagtttcaaaggcagttactcagttgagctcagtgctttcacctctgacttataagcccgcctacggaccctttaaacccaatgattccggataacgctcgcatcctccgtattaccgcggctgctggcacggagttagccgatgcttattcgtatggtaccgtcatcaatctacacgtagaccttattcttcccatacaaaagcagtttacaacccataaggccgtcatcctgcacgcggcatggctggatcagtcttccgaccattgtccaatattcctca

SEQ ID NO: 2 - DNA, 424 bp, synthetic construct
  misc_feature 1..424: note = Description of sequence: operational taxonomic unit; OUT#: denovo4616
  source 1..424: mol_type = other DNA
cctgttcgctccccacgctttcgtccatcagcgtcaatcggtcgttagtaacatgccttcgcaatcggtgttctgtgtaatatctatgcatttcaccgctacactacacattccagctacttcacaactattcaagacatacagtttcaaaggcagttactcagttgagctcagtgctttcacctctgacttatacgcccgcctacggaccctttaaacccaatgattccggataacgctcgcatcctccgtattaccgcggctgctggcacggagttagccgatgcttattcatacagtaccgtcatcaatctactcgtagaccttattcttcctgtataaaagcagtttacaacccataaggccgtcatcctgcacgcggcatggctggatcagtcttccgaccattgtccaatattcctca

SEQ ID NO: 3 - DNA, 429 bp, synthetic construct
  misc_feature 1..429: note = Description of sequence: operational taxonomic unit; OUT#: denovo5402
  source 1..429: mol_type = other DNA
cctgtttgctccccacgctttcgcacatgagcgtcagtacattcccaaggggctgccttcgccttcggtattcctccacatctctacgcatttcaccgctacacgtggaattctacccctccctaaagtactctagactcccagtctgaaatgcagttcccaagttaagctcggggatttcacatctcacttaaaagtccgcctgcgtgccctttacgcccagttattccgattaacgctcgcaccctccgtattaccgcggctgctggcacggagttagccggtgcttcttctgtaattaacgtcaatgatattatctattcaataataccccttcctcattaccgaaagaactttacaacccgaaggccttcttcattcacgcggcatggctgcgtcagggttccccccattgcgcaatattcccca

SEQ ID NO: 4 - DNA, 424 bp, synthetic construct
  misc_feature 1..424: note = Description of sequence: operational taxonomic unit; OUT#: denovo7264
  source 1..424: mol_type = other DNA
cctgttcgctccccacgctttcgtccatcagcgtcaataatttgttagtaacatgccttcgcaatcggtgttctgtgtaatatctatgcatttcaccgctacactacacattccagctacttcacaaatattcaagacctacagtttcaaaggcagttactcagttgagctcagtgctttcacctctgacttataagcccgcctacagaccctttaaacccaatgattccggataacgctcgcatcctccgtattaccgcggctgctggcacggagttagccgatgcttattcatacagtaccgtcatcaatctactcgtagatcttattcttcccgtataaaagcagtttacaacccataaggccgtcatcctgcacgcggcatggctggatcagtcttccgaccattgtccaatattcctca

SEQ ID NO: 5 - DNA, 424 bp, synthetic construct
  misc_feature 1..424: note = Description of sequence: operational taxonomic unit; OUT#: denovo13263
  source 1..424: mol_type = other DNA
cctgttcgctccccacgctttcgtccatcagcgtcagttgtaacttggtaacctgccttcgcaattggtgttctgagtaatatctatgcatttcaccgctacactactcattccagctacctcaaatacactcaagaccgacagtatcaacggcagttctacagttaagctgcaggctttcaccactgacttatcgacccgcctacggaccctttaaacccaataaatccggataacgcttgcaccctccgtattaccgcggctgctggcacggagttagccggtgcttattcatacagtaccttcagctaccctcacgagagtaggtttatccctgtataaaagaagtttacaaaccataggtccttagtccttcacgcgggatggctggatcaggctctcacccattgtccaatattcctca

SEQ ID NO: 6 - DNA, 429 bp, synthetic construct
  misc_feature 1..429: note = Description of sequence: operational taxonomic unit; OUT#: denovo479
  source 1..429: mol_type = other DNA
cctgtttgctacccacgctttcgaacctcagtgtcagtatgatgccaggaagctgccttcgccatcggtattcctccagatctctacgcatttcaccgctacacctggaattctacttccctctcacctactctagttacccagtatcagatgcagttcccaggttaagcccggggctttcacatctgacttaagtaaccacctacgctcgctttacgcccagtaattccgattaacgcttgcaccctctgtattaccgcggctgctggcacagagttagccggtgcttattctgtgggtaacgtcagggctaataggtattaattattagcttttcctccccacttaaagtgctttacaaccaaaaggccttcttcacacacgcggcatggctggatcaggctttcgcccattgtccaatattcccca

SEQ ID NO: 7 - DNA, 421 bp, synthetic construct
  misc_feature 1..421: note = Description of sequence: operational taxonomic unit; OUT#: denovo3045
  source 1..421: mol_type = other DNA
cctgttcgctccccacgctttcgctcctcagcgtcagtaacggcccagagacccgccttcgccaccggtgttcctcctgatatctgcgcattccaccgctacaccaggaattccagtctcccctaccgcactcaagccagcccgtacccaccgcaagcccacagttaagctgtgggttttcacggcagacgcgacaagccgcctacaagccctttacgcccaataattccggacaacgctcgcgccctacgtattaccgcggctgctggcacgtagttagccggcgcttctttacccactaccctcaactagaacaaaaactagccttgaccatgagcgaaagaggtttacaacccgaaggccgtcatccctcacgcggcgtcgctgcatcaggctttcgcccattgtgcaaaattcccca

SEQ ID NO: 8 - DNA, 407 bp, synthetic construct
  misc_feature 1..407: note = Description of sequence: operational taxonomic unit; OUT#: denovo9664
  source 1..407: mol_type = other DNA
cctgtttgctacccacgctttcgcgctttagcgtcagtatctgtccagtaagctggcttccccatcggcattcctacaaatatctacgaatttcacctctacacttgtagttccgcttacctctccagtactctagctatacagtttccaacgcaatacggagttgagccccgcattttcacatcagacttatttagccgcctagacgcgctttacgcccaataaatccggataacgcttgcgacatacgtattaccgcggctgctggcacgtatttagccgtcgcttcttctgttggtaccgtcactttcttcttcccaactgaaagcactttacattccgaaaaacttcatcgtgcacacagaattgctggatcagactcttggtccattgtccaatattcccca

SEQ ID NO: 9 - DNA, 429 bp, synthetic construct
  misc_feature 1..429: note = Description of sequence: operational taxonomic unit; OUT#: denovo9915
  source 1..429: mol_type = other DNA
cctgtttgctacccacgctttcgggcatgaacgtcagtattatcccagggggctgccttcgccatcggtattcctccacatctctacgcatttcactgctacacgtggaattctacccccctctgacatactctagctacccagtttcaaacgcagttcccaggttgagcccggggatttcacatctgacttaagtaaccgtctgcgcccgctttacgcccagtaattccgattaacgctcgcaccctacgtattaccgcggctgctggcacgtagttagccggtgcttattcttcaggtaccgtcatcactcaagggtattagcctcaagcttttcttccctgacaaaagtcctttacaacccgaaggccttcttcagacacgcggcatggctggatcaggcttgcgcccattgtccaaaattcccca

SEQ ID NO: 10 - DNA, 404 bp, synthetic construct
  misc_feature 1..404: note = Description of sequence: operational taxonomic unit; OUT#: denovo5773
  source 1..404: mol_type = other DNA
cctgtttgctccccacgctttcgtgcctcagcgtcagtttcagtccagaaagccgccttcgccaccggtgttcctcctaatatctacgcatttcaccgctacactaggaattccgctttcctctcctgtactctagtaagatagtttccaatgcacccctggagttgagccccagtctttcacaccagacttaccttaccgcctacgcaccctttacacccagtcattccggataacgcttgccccctatgtattaccgcggctgctggcacatagttagccggggcttcttagtcaggtaccgtcattttcttccctgctgatagaagtttacaatccgaagaccttcttccttcacgcggcattgctgcatcagagtttcctccattgtgcaatatccccca

SEQ ID NO: 11 - DNA, 405 bp, synthetic construct
  misc_feature 1..405: note = Description of sequence: operational taxonomic unit; OUT#: denovo6257
  source 1..405: mol_type = other DNA
cctgtttgctccccaaactgtcgtccctcatcgtcaagtattctgtagttagctgctttcgctttcggcgttccttccgatatcaacgcatttcaccgctccaccggaaattccactaacccccagatacttccagaattgcagtatctattccctttctacgattgagtcgcaggatttgaaaacagacttacaattcagactacggacactttacgcccagtaaatccggataacgcttgagacctacgtattaccgcggctgctggcacgtagttagccgtctcttattcttgacataccgtcattcgtcttctgccaaaaaagaagtttacacaccgtaatgcttcatccttcacgcgaaattgctgcatcagactttcgtccattgtgcaagattcccca

SEQ ID NO: 12 - DNA, 429 bp, synthetic construct
  misc_feature 1..429: note = Description of sequence: operational taxonomic unit; OUT#: denovo2026
  source 1..429: mol_type = other DNA
cctgtttgctccccacgctttcgtgcatgagcgtcagtgttatcccaggaggctgccttcgccatcggtgttcctccgcatatctacgcatttcactgctacacgcggaattccacctccctctgacacactctagccttgcagtcaccaatgcagttcccaggttaagcccggggatttcacatcggtcttgcaaaaccgcctgcgcactctttacgcccagtaattccgattaacgcttgcaccctacgtattaccgcggctgctggcacgtagttagccggtgcttattcttcaggtaccgtcatcggcccatggtattagcacggaccttttcgttcctgacaaaagtgctttacaacccgaaggccttcatcacacacgcggcattgctggatcaggcttgcgcccattgtccaaaattcccca

SEQ ID NO: 13 - DNA, 424 bp, synthetic construct
  misc_feature 1..424: note = Description of sequence: operational taxonomic unit; OUT#: denovo864
  source 1..424: mol_type = other DNA
cctgtttgatacccacgctttcgtgcttcagtgtcagttgcggtttggtaagctgccttcgcaatcggagttctgcgtaatatctatgcatttcaccgctacactacgcgttccgcctacctcatccgcactcaagctccacagtttcaacggcaagactatggttgagccacagcttttcaccactgacttatgaagccacctacgcaccctttaaacccaataaatccggataacgctcgtatcctccgtattaccgcggctgctggcacggagttagccgatacttattcttaggttacttgcttacagctacacgtaactgcttgtactcacctactaaagaagtttacaatccataggaccgtcttccttcacgcgacttggctggttcaggctctcgcccattgaccaatattcctca

SEQ ID NO: 14 - DNA, 429 bp, synthetic construct
  misc_feature 1..429: note = Description of sequence: operational taxonomic unit; OUT#: denovo2993
  source 1..429: mol_type = other DNA
cctgtttgctccccacgctttcgcacatgagcgtcagtatattcccaaggggctgccttcgccttcggtattcctccacatctctacgcatttcaccgctacacgtggaattctacccctccctaaaatactctagattgccagtctgaaatgcaattcccaggttaagcccggggctttcacatctcacttaacaatccgcctgcgtgccctttacgcccagtcattccgattaacgctcgcaccctccgtattaccgcggctgctggcacggagttagccggtgcttcttctgtggataacgtcaattaccaactctattaaagttgataccttcctcaccaccgaaagaactttacaacccgaaggccttcttcattcacgcggcatggctgcatcagggttccccccattgtgcaatattcccca

SEQ ID NO: 15 - DNA, 424 bp, synthetic construct
  misc_feature 1..424: note = Description of sequence: operational taxonomic unit; OUT#: denovo10732
  source 1..424: mol_type = other DNA
cctgtttgatacccacactttcgagcctcagcgtcagttacaccccagacagctgccttcgcaataggagttcttcgtgatatctaagcatttcaccgctacaccacgaattccaccatccttatgtgcactcaagaatatcagtatcaactgcaattttaaggttgagccccaaactttcacaactgacttaatatcccgcctacgctccctttaaacccaataaatccggataacgcccgaaccttccgtattaccgcggctgctggcacggaattagccggtcctttttcttatgatacttgcaaacagccacacgtggccgactttactctcatataaaagcagtttacaacccagaaggccgtcatcctgcacgctacttggctggttcagacttccgtccattgaccaatattcctca

SEQ ID NO: 16 - DNA, 424 bp, synthetic construct
  misc_feature 1..424: note = Description of sequence: operational taxonomic unit; OUT#: denovo2652
  source 1..424: mol_type = other DNA
cctgtttgatacccacactttcgagcctcagcgtcagtagcaccccggacacctgccttcgcgatcggagttcatcgtgatatctaagcatttcaccgctacaccacgagttccagcatccctgtgtgcactcaagactaccagtttcaacggcaatgtatggttgagccacacgctttaaccgctgacttaatagcccgcctacgctccctttaaacccaataaatccggataacgcctggaccttccgtattaccgcggctgctggcacggaattagccggtcctttttctgatggtacattcttatcagccacacgtggctgccgttattcccaactaaaagcagtttacaacccggagggccgtcatcctgcacgctacttggctggttcagactttcgtccattgaccaatattcctca

SEQ ID NO: 17 - DNA, 429 bp, synthetic construct
  misc_feature 1..429: note = Description of sequence: operational taxonomic unit; OUT#: denovo11999
  source 1..429: mol_type = other DNA
cctgtttgctccccacgctttcgtgcatgagcgtcagtacaggcccaggggactgccttcgccatcggtgttcctccgcatatctacgcatttcactgctacacgcggaattccatccccctctgccgtactccagccctgcagtcacaaaggcagttcccaggttgagcccggggatttcacctctgtcttacagaaccgcctgcgcacgctttacgcccagtaattccgattaacgctcgcaccctacgtattaccgcggctgctggcacgtagttagccggtgcttattcttacagtaccgtcatgatccccagatattagccagagacttttcgttctgtacaaaagcagtttacaacccgaaggccttcatcctgcacgcggcattgctggatcaggcttgcgcccattgtccaaaattcccca

SEQ ID NO: 18 - DNA, 429 bp, synthetic construct
  misc_feature 1..429: note = Description of sequence: operational taxonomic unit; OUT#: denovo11474
  source 1..429: mol_type = other DNA
cctgtttgctccccacgctttcgtgcctcagtgtcagtgctggcccagatagccgccttcgccacggatgttcctcctgatctctacgcatttcactgctacaccaggaattccgctatcctctaccgcactctagcttgccagtatcaaatgcagttcccaggttgagcccagggctttcacatctgacttaacaaaccacctacgcacgctttacgcccagtaattccgagtaacgcttgcacccttcgtattaccgcggctgctggcacgaagttagccggtgcttattctttgggtaccgtcatgacacacgggtattaaccgcatgcttttctttcccaacaaaagggctttacaacccgaaggccttcttcacccacgcggcatggctggatcaggcttgcgcccattgtccaatattcccca

SEQ ID NO: 19 - DNA, 428 bp, synthetic construct
  misc_feature 1..428: note = Description of sequence: operational taxonomic unit; OUT#: denovo9983
  source 1..428: mol_type = other DNA
cctgtttgctccccacgctttcgtccctcagcgtcagtatagacccagtaagctgccttcgcctttggtgttcttccatatatctacgcatttcaccgcttcacatggaattccgcttacctctatccaactctagtttgccagtatccaaagcgagccggggttgagccccgggatttgactccagacttaacaaacagcctacgaacgctttacgcccaataattccggataacgcttgcgacctatgtattaccgcggctgctggcacatagttagccatcgctttctgacaaggtaccgtcagcctaatagcatttcctctactaggtgttcttcccttataacagcactttacaatccgaagaccttcatcgtgcacgctgtgtcgctccatcaagctttcgctcattgtggaatattcccta

SEQ ID NO: 20 - DNA, 408 bp, synthetic construct
  misc_feature 1..408: note = Description of sequence: operational taxonomic unit; OUT#: denovo1688
  source 1..408: mol_type = other DNA
ccgttttgctacccacgctttcgagccttagtgtcagacaaggaccagcgaatcgccttcgccactgatgttcctcccaatatcaacgcatttcaccgctccactgggaattccattcgcctcttcctgtctcaattcctgtagtttccaaagctttcccacggttgagccgtggtctttaactaaagacttacagaaacacctacgcatctctatacacccaataaatccggataacgttcgcaccctacgcatcaccgatgcttctggcacgtagttagcaggtgcttattcatatggtaccgtcatctattcttcccatataaaaggagtttacaatccgaagaccgtcatcctccacactgtgtcgctgcgtcagggttgcccccattgcgcaagattcctaat

SEQ ID NO: 21 - DNA, 429 bp, synthetic construct
  misc_feature 1..429: note = Description of sequence: operational taxonomic unit; OUT#: denovo13493
  source 1..429: mol_type = other DNA
cctgttcgctacccacgctttcgagcctcagcgtcagttacagaccagagagccgctttcgccaccggtgttcctccatatatctacgcatttcaccgctacacatggaattccactctccccttctgcactcaagtttaacagtttccaaagcatactatggttaagccacagccttttacttcagacttatcaaaccgcctgcgctcgctttacgcccaataaatccggacaacgctcgggacctacgtattaccgcggctgctggcacgtagttagccgtccctttctggtcagttaccgtcactatgtgaactttccactctcacatacattcttggctgacaacagagctttacgatccgaaaaccttcttcactcacgcggcgttgctcggtcagggttgcccccattgccgaagattcccta

SEQ ID NO: 22 - DNA, 425 bp, synthetic construct
  misc_feature 1..425: note = Description of sequence: operational taxonomic unit; OUT#: denovo5825
  source 1..425: mol_type = other DNA
cctgtttgatacccacgctttcgtgctttagtgtcagatgaagtctggtacgctgccttcgcaattggcgttctgtgtgatatctatgcatttcaccgctacaccacacattccacatacctcgtcttctctcgagtccttcagtttcaacggcaagactacagttgagctgcagcttttcaccactgacttaaaaaaccacctacacaccctttaaacccaataattccggataacgctcgtatcctccgtattaccgcggctgctggcacggagttagccgatacttattcgtagagtacatgcatctcggttactcgtaaccgccattattcctctacaaaagaagtttacaatccatagaaccgtcttccttcacgcgacttggctggttcaggctttcgcccattgaccaatattcctca

SEQ ID NO: 23 - DNA, 429 bp, synthetic construct
  misc_feature 1..429: note = Description of sequence: operational taxonomic unit; OUT#: denovo4000
  source 1..429: mol_type = other DNA
cctgttcgatccccacactttcgcatctgagcgtcaattatcggccagaaacccgccttcgccaccggtgttcttccaaatatctacagattccacccctacacttggaattccagtttcccttccgtaattcaagttatgcagtacccaatgctgttctgaggttaagcctcaggatttcacatcaggcttacataaccgcctgcatgccctttacgcccaataattccgaacaacgctcgccccttacgtgttaccgcggctgctggcacgtaattagccggggcttattcgccgattaccgtcatctgataagcattccctcttatcattattcttcatcggcaaaaggattttacaacttttcagccttcttcatccacgcggcgtcgctccgtcagacttccgtccattgcggaatattcttag

SEQ ID NO: 24 - DNA, 429 bp, synthetic construct
  misc_feature 1..429: note = Description of sequence: operational taxonomic unit; OUT#: denovo15463
  source 1..429: mol_type = other DNA
cctgttcgctccccgcactttcgcacctcagcgtcaattatctgccggtaacctgccttcgccattggtattcttccacatatctacagatttcacccctacacgtggaattctggttacccttcagtaattcaagatgcgcagttctcaatgcagttccgcagttaagctgcagtatttcacaccaagcttgcatatccgcctacatgccctttacgcccaataattccgaacaacgctcgcaacttacgtgttaccgcggctgctggcacgtaattagccgttgcttattcaagccctacagtcaccgcagaggcattccctcctccgcttattcctcaggcttaaaaggattttacaacctttcggcattcttcatccacgcggcgtcgctccgtcagactttcgtccattgcggaatattcttaa

SEQ ID NO: 25 - DNA, 430 bp, synthetic construct
  misc_feature 1..430: note = Description of sequence: operational taxonomic unit; OUT#: denovo10545
  source 1..430: mol_type = other DNA
cctgtttgctccccgcaccttcgcatctcagcgtcaatcatcggccagaaacccgccttcgccaccggtgttcttccaaatatctacagattccacccctacacttggaattccggtttcccctccgtgattctagtttagcagtacccaatgcagtttacgagttaagctcgtagatttcacatcaggcttaccaaaccgcctacatgccctttacgcccaataattccgaacaacgctcgccccttacgtgttaccgcggctgctggcacgtaattagccggggcttattcgcatgactaccgtcatctcacaggcattccctcccgtgattattcttcgtctgcaaaagaattttacaacttttcagccttcttcattcacacggcgtcgctccgtcagactttcgtccattgcggaagattcttag

SEQ ID NO: 26 - DNA, 403 bp, synthetic construct
  misc_feature 1..403: note = Description of sequence: operational taxonomic unit; OUT#: denovo6529
  source 1..403: mol_type = other DNA
cctgtttgctacccacgctttcgtgcctcagtgtcagttgcagtccagaaagccgccttcgctactggtgttcctcccaatatctacgcatttcaccgctacactgggaattccactttcctctcctgcactcaagtctaacagtttcggaggcttactacggttgagccgtagcctttaaccaccgacttgaaagaccacctacgcaccctttacgcccagtaaatccggataacgctagccccctacgtattaccgcggctgctggcacgtagttagccggggcttcctcacagggtaccgtcattatcttcccctgcaacagaactttacgatccgaagaccttcatcgttcacgcggcgttgctgcatcaggcttgcgcccattgtgcaatattcccca

SEQ ID NO: 27 - DNA, 404 bp, synthetic construct
  misc_feature 1..404: note = Description of sequence: operational taxonomic unit; OUT#: denovo652
  source 1..404: mol_type = other DNA
cctgtttgctccccacgctttcgagcctgagcgtcagttatcgtccagtaagccgccttcgccaccgatgttcctcctaatatctacgcatttcaccactacaccaggaattccgcttacctctccgacactctagcttgacagtttccaatgcaaccccggagttaagccccggtctttcacatcagacttgccatgccgcctacgctccctttacgcccagtaaatccggataacgcttgccccatacgtattaccgcggctgctggcacgtatttagccagggcttcttagtcaggtaccgtcattttcttccctgctgatagaagtttacataccgaaatacttcatccttcacgcggcgtcgctgcatcaggctttcgcccattgtgcaatatccccca

SEQ ID NO: 28 - DNA, 406 bp, synthetic construct
  misc_feature 1..406: note = Description of sequence: operational taxonomic unit; OUT#: denovo78
  source 1..406: mol_type = other DNA
cccgtttgctccctacgctttcgtgccttagtgtcagaatagtcccagtaacctgcctacgctatcggtgttccttctaatatctacggatttcactcctacactagaaattccagttacccatgactatctcgagcttagcagtttagataatagtctgaatggttgagccaccagatttcactatctacttactatgccacctacgcaactctttacacccagtcactccggataatgctcggatcctacgtatgaccgcggctgctggcacgtagttagccgatccttattcacaagttaccatcatatttctcgcttgtaaaagcagtttacaacccgaaggccttcatcctgcacgcggcgttgctccatcaggctttcgcccattgtgaaagattcccta

SEQ ID NO: 29 - DNA, 430 bp, synthetic construct
  misc_feature 1..430: note = Description of sequence: operational taxonomic unit; OUT#: denovo8534
  source 1..430: mol_type = other DNA
cctgtttgctccccgcaccttcgcatctcagcgtcaatcatcagccagaaacccgccttcgccaccggtgttcttccaaatatctacagattccacccctacacttggaattccggtttcccctctgtgattccagtttagcagtacccaatgcagtttgcgagttaagctcgcagatttcacatcaggcttaccaaaccgcctacatgccctttacgcccaataattccgaacaacgctcgccccttacgtgttaccgcggctgctggcacgtaattagccggggcttattcgcatgactaccgtcatcagataggcattccctcctatccttattcttcatctgcaaaagaattttacaacttttcagccttcttcattcacacggcgtcgctccgtcagactttcgtccattgcggaagattcttag

SEQ ID NO: 30 - DNA, 429 bp, synthetic construct
  misc_feature 1..429: note = Description of sequence: operational taxonomic unit; OUT#: denovo11699
  source 1..429: mol_type = other DNA
cctgttcgatccccacactttcgcatctgagcgtcaattatcggccagaaacccgccttcgccaccggtgttcttccaaatatctacagattccacccctacacttggaattccagtttcccttccgtaattcaagtcgtgcagtacccaatgctattctgaggttaagcctcaggatttcacatcaggcttacacaaccgcctgcatgccctttacgcccaataattccgaacaacgctcgccccttacgtgttaccgcggctgctggcacgtaattagccggggcttattcgctgattaccgtcatcagataagcattccctcttacctttattcttcatcaacaaaaggattttacaacttttcagccttcttcatccacgcggcgtcgctccgtcagacttccgtccattgcggaatattcttag

SEQ ID NO: 31 - DNA, 428 bp, synthetic construct
  misc_feature 1..428: note = Description of sequence: operational taxonomic unit; OUT#: denovo10845
  source 1..428: mol_type = other DNA
cccgttcgctaccctggctttcgagcctcagtgtcagttgccgtccagaaagccgccttcgccactggtgttcttcccaatatctacgcatttcaccgctacactgggaattccgctttcctctccggcactcaagagttgtagtttctttcccatcacggggctgagccccgaacttttaagaaagacttacatccccacctgcgctccctttacgcccaataattccggacaacgcttgccgcctacgtattaccgcggctgctggcacgtagttagccgtggcttcctcgtcaggtaccgtcttagaccagccattgcctgctgctccttttctcccctgacaacagagttttacgatccgaagaccttcttcactcacgcggcgttgctccgtcaggctttcgcccattgcggaagattcccca

SEQ ID NO: 32 - DNA, 429 bp, synthetic construct
  misc_feature 1..429: note = Description of sequence: operational taxonomic unit; OUT#: denovo9061
  source 1..429: mol_type = other DNA
cccgttcgctcccctggctttcgagcctcagcgtcagttacagtccagaaagccgccttcgccactggtgttcctcccaatatctacgcatttcaccgctacactgggaattccgctttcctctcctgcactcaaggtatacagtttccatcccctcacggggttgagccccgcacttttaagatggactgatatatccgcctgcgctccctttacgcccaataattccggacaacgctcgccacctacgtattaccgcggctgctggcacgtagttagccgtggcttcctcgctcggtaccgtcacccgggttcaatattttcaaacccggttttcgtcccgtgcaacagagctttacgatccgaagaccttcttcactcacgcggcgttgctccatcaggctttcgcccattgtggaagattcccca

SEQ ID NO: 33 - DNA, 430 bp, synthetic construct
  misc_feature 1..430: note = Description of sequence: operational taxonomic unit; OUT#: denovo5093
  source 1..430: mol_type = other DNA
cctgtttgctccccgcaccttcgcatatcagcgtcaatcatcggccagaaacccgccttcgccaccggtgttcttccaaatatctacagattccacccctacacttggaattccggtttcccctccgtgattcaagttaagcagtacccaatgcagtttacgagttaagctcgtagatttcacatcaggcttacctaaccgcctacatgccctttacgcccaataattccgaacaacgcttggggcttacgtgttaccgcggctgctggcacgtaattagccgccccttattcgcatgattaccgtcatcagataggcattccctcctatccttattcttcatctgcaaaagaactttacaacctttcggccttcatcgttcacgcggcgtcgctccgtcagactttcgtccattgcggaagattcttag

SEQ ID NO: 34 - DNA, 429 bp, synthetic construct
  misc_feature 1..429: note = Description of sequence: operational taxonomic unit; OUT#: denovo329
  source 1..429: mol_type = other DNA
cctgttcgatccccacactttcgcacctcagcgtcaattatcggccagaaacccgccttcgccaccggtgttcttccaaatatctacagattccacccctacacttggaattccagtttcccttccgtaattcaagttatacagtacccaatgctatcctagggttaagccccagcatttcacatcaggcttacataaccgcctacatgccctttacgcccaataattccgaacaacgctcgccccttacgtgttaccgcggctgctggcacgtaattagccggggcttattcgtcgactaccgtcatcaggtgagcattccctctcacccttattcttcatcaacaaaaggattttacaacttttcagccttcttcatccacgcggcgtcgctccgtcagacttccgtccattgcggaatattcttag

SEQ ID NO: 35 - DNA, 430 bp, synthetic construct
  misc_feature 1..430: note = Description of sequence: operational taxonomic unit; OUT#: denovo10686
  source 1..430: mol_type = other DNA
cctgtttgctccccgcaccttcgcaccttagcgtcaatcattggctagaaacccgccttcgccaccggtgttcttccaaatatctacagattccacccctacacttggaattccagtttcccctccatgattcaagtcaagcagtacccaatgcagtttacgagttaagctcatacatttcacatcaggcttacctaaccgcctacatgccctttacgcccaataattccgaacaacgctcgccccttacgtattaccgcggctgctggcacgtaattagccggggcttattcgcataactaccgtcatcagatagtcatttccttctatccttattctttgtctgcaaaagaactttacaaccttccggccttcatcgttcacgcggtgtcgctccgtcagactttcgtccattgcggaagattctcag

SEQ ID NO: 36 - DNA, 430 bp, synthetic construct
  misc_feature 1..430: note = Description of sequence: operational taxonomic unit; OUT#: denovo7737
  source 1..430: mol_type = other DNA
cctgtttgctacccacgctttcgtgcctcagcgtcaatatcgggccaggtagccgccttcgccaccggtgttcctcctgatatctacggatttcactcctactccaggaattccgctaccctctcccgaattccagttctgaagtttcaaatgcagtgcctcggttgagccgagaaatttcacacctgactttcaaaacagcctacgcaccctttacgcccagtaattccgaataacgcttgcaccctccgtattaccgcggctgctggcacggagttagccggtgcttcctctggaggtaccgtcaaatcccatggctattaaccataaaacacttcttccctcctgacaggggtttacaatccgaaaaccttcttcccccacgcggcgttgctgcgtcaggctttcgcccattgcgcaatattcccca

SEQ ID NO: 37 - DNA, 430 bp, synthetic construct
  misc_feature 1..430: note = Description of sequence: operational taxonomic unit; OUT#: denovo4245
  source 1..430: mol_type = other DNA
cctgtttgctccccgcaccttcgcatctcagcgtcaatcatcggccagaaacccgccttcgccaccggtgttcttccaaatatctacagattccacccctacacttggaattccggttccccctccgtgattcaagttaagcagtacccaatgcagtttacgagttaagctcgtagatttcacatcaggcttacctaaccgcctacatgccctttacgcccaataattccgaacaacgctagaggcttacgtgttaccgcggctgctggcacgtaattagccgccccttattcgcatgactaccgtcatcagacaagcattccctcttgcccttattcttcatctgcaaaagaactttacaacctttcggccttcttcattcacacggcgtcgctccgtcagactttcgtccattgcggaagattcttag

SEQ ID NO: 38 - DNA, 403 bp, synthetic construct
  misc_feature 1..403: note = Description of sequence: operational taxonomic unit; OUT#: denovo8371
  source 1..403: mol_type = other DNA
cctgtttgctccccgcgctttcgtacctcagcgtcagttaaagtccagaaagtcgccttcgccaccggtattcctcctgatatctacgcatttcaccgctacaccaggaattccactttcctctccttcactcaagtctatcagtttcatatgctttatagggttaagccctacgctttaacatctgacttcttagaccgcctgcgtaccctttacgcccaataattccggacaacgctcgccccctacgtattaccgcggctgctggcacgtagttagccggggcttcctcctatggtaccgtcattatctttccataggacagagctttacgaaacgaattccttcttcgctcacgcggcgtcgctgcatcagggttccccccattgtgcaaaattcccca